SEQUENCE LISTING (1 sequence)

SEQ ID NO: 1 - DNA, 3324 bp, Artificial Sequence
  synthetic modified self-vector BHT-3021 with BHT-1 expression vector backbone and polynucleotide encoding human proinsulin
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtaagtatcaaggttacaagacaggtttaaggagaccaatagaaactgggcttgtcgagacagagaagactcttgcgtttctgataggcacctattggtcttactgacatccactttgcctttctctccacaggcttaagcttatggcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgcggccaaggatctgatggcgcaggggatcaagctctgatcaagagacaggatgaggatggtttcgcatgattgaacaagatggattgcacgcaggttctccggcagcttgggtggagaggctattcggctatgactgggcacaacagacaatcggctgctctgatgccgccgtgttcaggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggcaggttttctggattcatcgactgtggccggctgggtgtggcggacaggtatcaggacatagcgttggctacccgtgatattgctgaagagcttggcggcgaatgggctgacaggttcctcgtgctttacggtattgcggctcccgattcgcagcgcattgccttctataggcttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2018/0092991 A1
(43) Pub. Date: Apr. 5, 2018

(54) COMPOSITIONS AND METHODS FOR TREATMENT OF INSULIN-DEPENDENT DIABETES MELLITUS

(71) Applicants: Hideki Garren, Palo Alto, CA (US); Michael Leviten, Palo Alto, CA (US); Nanette Solvason, Palo Alto, CA (US)

(72) Inventors: Hideki Garren, Palo Alto, CA (US); Michael Leviten, Palo Alto, CA (US); Nanette Solvason, Palo Alto, CA (US)

(73) Assignee: BAYHILL THERAPEUTICS, INC., Palo Alto, CA (US)

(21) Appl. No.: 13/376,345

(22) PCT Filed: Jun. 7, 2010
(86) PCT No.: PCT/US2010/037630

§ 371 Date: Nov. 11, 2015

Related U.S. Application Data
(60) Provisional application No. 61/184,616, filed on Jun. 5, 2009.

(51) Int. Cl. A61K 48/00; C07K 14/47; C07K 14/62; C12N 15/86; A61K 9/00; A61P 3/10

(52) U.S. Cl. CPC A61K 48/0066; C07K 14/4713; C07K 14/62; C12N 2830/001; A61K 9/0019; A61P 3/10; C12N 15/86

(57) ABSTRACT

This invention provides methods of treating insulin-dependent diabetes mellitus in a subject comprising administering to the subject a self-vector encoding human proinsulin. The invention also provides a pharmaceutical composition comprising a self-vector encoding human proinsulin, as well as treatment and maintenance regimens for administering the pharmaceutical composition to a subject.

### BACKGROUND OF THE INVENTION

Autoimmune disease is a disease caused by adaptive immunity that becomes misdirected at healthy cells and/or tissues of the body. Autoimmune diseases are characterized by T and B lymphocytes that aberrantly target self-proteins, -polypeptides, -peptides, and/or other self-molecules causing injury and or malfunction of an organ, tissue, or cell-type within the body to cause the clinical manifestations of the disease (Marrack et al., Nat Med 7, 899-905, 2001). Autoimmune diseases include diseases that affect specific tissues as well as diseases that can affect multiple tissues. For tissue-specific autoimmune diseases, the characteristic feature is the selective targeting of a single tissue or individual cell type.

Human type I or insulin-dependent diabetes mellitus (IDDM) is a tissue-specific autoimmune disease characterized by autoimmune destruction of the β cells in the pancreatic islets of Langerhans. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2).

Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration. Blood C-peptide concentrations can also be measured as an indicator of beta cell function. A decrease of blood C-peptide levels is indicative of disease.

The presence of combinations of autoantibodies with various specificities in serum are highly sensitive and specific for human type I diabetes mellitus. For example, the presence of autoantibodies against GAD and/or IA-2 is approximately 98% sensitive and 99% specific for identifying type I diabetes mellitus from control serum. In non-diabetic first degree relatives of type I diabetes patients, the presence of autoantibodies specific for two of the three autoantigens including GAD, insulin and IA-2 conveys a positive predictive value of >90% for development of type IDM within 5 years.

Autoantigens or self-proteins targeted in human insulin dependent diabetes mellitus include, for example, insulin autoantigens, including insulin, insulin B chain, proinsulin, and preproinsulin; tyrosine phosphatase IA-2; IA-2β; glutamic acid decarboxylase (GAD) both the 65 kDa and 67 kDa forms; carboxypeptidase H; heat shock proteins (HSP); glima 38; islet cell antigen 69 KDa (ICA69); p52; two ganglioside antigens (GT3 and GM2-1); islet-specific glucose-6-phosphatase-related protein (IGRP); zinc transporter Slc30A8; and an islet cell glucose transporter (GLUT 2).

To treat human tissue-specific autoimmune diseases such as IDDM, a number of different therapeutic approaches have been tried. Soluble protein antigens have been administered systemically to inhibit the subsequent immune response to that antigen. In the case of human IDDM, recombinant insulin is delivered by injection or pump-based delivery (Pozzilli and Gisella Cavallo, Diabetes Metab Res Rev, 16:306-7 (2000). Another approach is the attempt to design rational therapeutic strategies for the systemic administration of a peptide antigen based on the specific interaction between the T cell receptors and peptides bound to MHC molecules. One study, using the peptide approach in an animal model of diabetes, resulted in the development of antibody production to the peptide (Hurtenbach, U. et al., J Exp. Med, 177:1499 (1993)). Another approach is the administration of T cell receptor (TCR) peptide immunization. See, e.g., Vandenbark, A. A. et al., Nature, 341:541 (1989). Still another approach is the induction of oral tolerance by ingestion of peptide or protein antigens. See, e.g., Weiner, H. L., Immmunol Today, 18:335 (1997).

Alternatively, immune responses can be altered by vaccination. Various approaches include delivering proteins, polypeptides, or peptides, alone or in combination with adjuvants (immunostimulatory agents); delivering an attenuated, replication deficient, and/or non-pathogenic form of a virus or bacterium; or delivering plasmid DNA. DNA vaccination, or polynucleotide therapy, is an efficient method to induce immunity against foreign pathogens (Davis, 1997; Hassett and Whitton, 1996; and Ulmer et al., 1996) and cancer antigens (Stevenson et al., 2004) and to modulate autoimmune processes (Waisman et al., Nat. Med, 2:899-905, 1996). Following intramuscular injection, plasmid DNA is taken up by, for example, muscle cells allowing for the expression of the encoded polypeptide (Wolff et al., 1992) and the mounting of a long-lived immune response to the expressed proteins (Hassett et al., 2000). In the case of autoimmune disease, the effect is a shift in an ongoing immune response to suppress autoimmune destruction and is believed to include a shift in self-reactive lymphocytes from a Th1- to a Th2-type response. The modulation of the immune response may not be systemic but occur only locally at the target organ under autoimmune attack.

Methods for treating autoimmune disease by administering a nucleic acid encoding one or more autoantigens or self-proteins have been described, for example, in International Patent Application Nos. WO 00/53019, WO 2003/045316, and WO 2004/047734. While these methods have been successful, further improvements are still needed.

### BRIEF SUMMARY OF THE INVENTION

The present invention provides compositions and methods for treating insulin-dependent diabetes mellitus (IDDM) and/or related diseases in a subject comprising administration of a self-vector encoding and capable of expressing an autoantigen (also referred to as self-protein) associated with or targeted in IDDM and/or related diseases, for example, human proinsulin. Other autoantigens or self-proteins associated with or targeted in IDDM are known in the art and find use in the present invention. For example, a self-vector comprising a polynucleotide encoding one or more of insulin, insulin B chain, proinsulin, and preproinsulin; tyrosine phosphatase IA-2; IA-2β; glutamic acid decarboxylase (GAD) both the 65 kDa and 67 kDa forms; carboxypeptidase H; heat shock proteins (HSP); glima 38; islet cell antigen 69 KDa (ICA69); p52; two ganglioside antigens (GT3 and GM2-1); islet-specific glucose-6-phosphatase-related protein (IGRP); zinc transporter Slc30A8; and an islet cell glucose transporter (GLUT 2) can be administered according to the regimens and using for example the BHT-1 vector as described herein. The present invention also relates to the co-administration of a self-vector comprising a polynucleotide encoding one or more self proteins associated with or targeted in IDDM and/or related diseases and the self-proteins encoded by the self-vector.

Accordingly, in one aspect, the invention provides methods of reducing disease severity, for example, by slowing or stopping disease progression, in a subject afflicted with insulin dependent diabetes mellitus (IDDM), the method comprising administering intramuscularly to the subject a DNA plasmid vector or self-vector encoding a self-protein associated with or targeted in IDDM (e.g., proinsulin), wherein the administration of the DNA plasmid vector is according to a regimen comprising a combination of:

(a) a therapeutically effective amount of the DNA plasmid vector or self-vector, for example, SEQ ID NO:1 (BHT 3021), of from 0.3 to 6 mg;

(b) a dose frequency of weekly or bi-weekly dosing; and,

(c) a period of dosing selected from the group consisting of continuous dosing, four (4) weeks of dosing, six (6) weeks of dosing, twelve (12) weeks of dosing, twenty-four (24) weeks of dosing, one (1) year of dosing, eighteen (18) months of dosing or twenty-four (24) months (i.e., two (2) years) of dosing.

In some embodiments, a reduction in the severity of IDDM in the subject is indicated by one or more measures selected from the group consisting of increased or stabilized levels of C-peptide, decreased or stabilized levels of glycosylated hemoglobin, decreased hyperglycemia, decreased hypoglycemia, decreased variability in blood glucose, decreased use of exogenous insulin, increased plasma insulin, decreased glucosuria, decreased insulitis, decreased destruction of beta-cells, and decreased presence of autoantibodies.

In some embodiments, the subject is a human.

In some embodiments, the DNA plasmid vector or self-vector, for example, SEQ ID NO:1 (BHT 3021), is administered weekly. In some embodiments, the DNA plasmid vector is administered bi-weekly (i.e., once every other week or once every two weeks). In some embodiments, the DNA plasmid vector or self-vector is administered monthly.

In some embodiments, the period of dosing is continuously, i.e., weekly or bi-weekly, over the full period of treatment. In some embodiments, the period of dosing is continuously (e.g., weekly or bi-weekly for one year, weekly or bi-weekly for the life of the patient or weekly or bi-weekly until a desired therapeutic endpoint is reached). In some embodiments, the period of dosing is 4, 5, 6, 7, 8, 9, 10, 11 or 12 months, 1.5 years, or 2 years, or longer or shorter periods of time, as desired or necessary, e.g., to achieve a desired therapeutic effect. In some embodiments, the period of dosing is four (4) weeks (e.g., 2 or 4 administrations). In some embodiments, the period of dosing is six (6) weeks (e.g., 3 or 6 administrations). In some embodiments, the period of dosing is twelve (12) weeks (e.g., 6 or 12 administrations). In some embodiments, the period of dosing is 24 weeks (e.g., 12 or 24 administrations). In some embodiments, the period of dosing is one year (e.g., 26 or 52 administrations). In some embodiments, the period of dosing is 1.5 years or 18 months (e.g., 39 or 78 administrations). In some embodiments, the period of dosing is two years (e.g., 52 or 104 administrations).

In some embodiments, the therapeutic regimen further comprises a supplemental or maintenance regimen comprising administering a therapeutically effective amount of the DNA plasmid or self-vector at a subsequent dose frequency of weekly or bi-weekly dosing for a dosing period of six (6) weeks. In some embodiments, the therapeutic regimen and/or supplemental regimen are repeated once every six (6) months, once every nine (9) months or once per year.

In some embodiments, the regimen comprises administering a dose of 0.3 to 6 mg of the DNA plasmid vector or self-vector weekly for 12 weeks followed by administering a dose of 1 to 3 mg of the DNA plasmid vector or self-vector bi-weekly for 6 weeks; wherein the regimen is repeated once per year. In some embodiments, the dose of the DNA plasmid vector or self-vector is 0.3 mg, 1 mg, 2 mg, 3 mg or 6 mg.

In some embodiments, the regimen comprises administering a dose of 1 to 3 mg of the DNA plasmid vector or self-vector weekly for 12 weeks followed by administering a dose of 1 to 3 mg of the DNA plasmid vector or self-vector bi-weekly for 6 weeks; wherein the regimen is repeated once per year. In some embodiments, the dose of the DNA plasmid vector or self-vector is 1 mg, 2 mg or 3 mg.

In some embodiments, the regimen comprises administering a dose of 0.3 to 6 mg of the DNA plasmid vector or self-vector bi-weekly for the life of the patient, or until a therapeutic endpoint is reached and maintained. In some embodiments, the dose of the DNA plasmid vector or self-vector is 0.3 mg, 1 mg, 2 mg, 3 mg or 6 mg.

In some embodiments, the regimen comprises administering a dose of 1 to 3 mg of the DNA plasmid vector or self-vector bi-weekly for the life of the patient, or until a therapeutic endpoint is reached and maintained. In some embodiments, the dose of the DNA plasmid vector or self-vector is 1 mg, 2 mg or 3 mg.

In some embodiments, the regimen comprises administering a dose of 0.3 to 6 mg of the DNA plasmid vector or self-vector bi-weekly for 6 weeks followed by administering a dose of 0.3 to 6 mg of the DNA plasmid vector or self-vector monthly for the life of the patient. In some embodiments, the dose of the DNA plasmid vector or self-vector is 0.3 mg, 1 mg, 2 mg, 3 mg or 6 mg.

In some embodiments, the regimen comprises administering a dose of 1 to 3 mg of the DNA plasmid vector or self-vector bi-weekly for 6 weeks followed by administering a dose of 1 to 3 mg of the DNA plasmid vector or self-vector monthly for the life of the patient. In some embodiments, the dose of the DNA plasmid vector or self-vector is 1 mg, 2 mg or 3 mg.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 1 mg of the DNA plasmid vector or self-vector is administered bi-weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 2 mg of the DNA plasmid vector or self-vector is administered bi-weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered bi-weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 6 mg of the DNA plasmid vector or self-vector is administered bi-weekly for a period of four (4) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 6 mg of the DNA plasmid vector or self-vector is administered weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered bi-weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered bi-weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 3 mg of the DNA plasmid vector or self-vector is administered bi-weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered bi-weekly for a period of six (6) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 0.3 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 6 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 3 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 1 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 6 mg of the DNA plasmid vector or self-vector is administered bi-weekly for a period of twelve (12) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 0.3 mg of the DNA plasmid vector or self-vector is administered weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 1 mg of the DNA plasmid vector or self-vector is administered weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered bi-weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered bi-weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered bi-weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered bi-weekly for a period of twenty-four (24) weeks. In some embodiments, this therapeutic regimen is followed by a supplemental or maintenance regimen, as described herein. In some embodiments, this therapeutic and/or maintenance regimen is repeated once every six (6) months, once every nine (9) months or once per year.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 2 mg of the DNA plasmid vector or self-vector is administered weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for. example, SEQ ID NO: 1 (BHT 3021), wherein 1 mg of the DNA plasmid vector or self-vector is administered bi-weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered bi-weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered bi-weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered bi-weekly for a period of one (1) year. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 1 mg of the DNA plasmid vector or self-vector is administered weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 3 mg of the DNA plasmid vector or self-vector is administered weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 6 mg of the DNA plasmid vector or self-vector is administered weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered bi-weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 2 mg of the DNA plasmid vector or self-vector is administered bi-weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered bi-weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered bi-weekly for a period of eighteen (18) months. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered bi-weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered bi-weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered bi-weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO: 1 (BHT 3021), wherein 3 mg of the DNA plasmid vector or self-vector is administered bi-weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 6 mg of the DNA plasmid vector or self-vector is administered bi-weekly for a period of two (2) years. In some embodiments, this therapeutic regimen is followed by a supplemental or maintenance regimen, as described herein. In some embodiments, this therapeutic and/or maintenance regimen is repeated one, two, three, or more times, as needed or desired.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 2 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 3 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021) wherein 6 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 0.3 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector, for example, SEQ ID NO:1 (BHT 3021), wherein 1 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector of SEQ ID NO:1 (BHT 3021), wherein 2 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector of SEQ ID NO:1 (BHT 3021), wherein 3 mg of the DNA plasmid vector or self-vector is administered continuously. In some embodiments, this therapeutic regimen is followed by or intermittently exchanged with a supplemental or maintenance regimen, as described herein.

In a further aspect, the invention provides methods of reducing disease severity in a subject afflicted with insulin dependent diabetes mellitus (IDDM), the method comprising administering intramuscularly to the subject a DNA plasmid vector or self-vector of SEQ ID NO:1 (BHT 3021), wherein 6 mg of the DNA plasmid vector or self-vector is administered continuously. In some embodiments, this therapeutic regimen is followed by or intermittently exchanged with a supplemental or maintenance regimen, as described herein.

Further embodiments of the therapeutic and supplemental or maintenance regimes are as described herein.

In a further aspect, the invention provides a self-vector of SEQ ID NO:1 (BHT-3021).

In some embodiments, the invention provide compositions comprising a self-vector of SEQ ID NO: 1 (BHT-3021) and a pharmaceutically acceptable carrier or excipient.

In some embodiments, the composition further comprises calcium at a concentration about equal to physiological levels (e.g., about 0.9 mM).

In some embodiments, the composition further comprises a divalent cation at a concentration greater than physiological levels. In some embodiments, the divalent cation is calcium. In some embodiments, the calcium is at a concentration greater than about 2 mM; in some embodiments the calcium is at a concentration of about 5.4 mM. In some embodiments, the composition is endotoxin-free. In some embodiments, the pharmaceutically acceptable carrier comprises an adjuvant.

In a related aspect, the present invention provides methods of treating, preventing, reducing the severity of, and/or amelioriating the symptoms of insulin-dependent diabetes mellitus (IDDM) in a subject comprising administering to the subject a self-vector of SEQ ID NO:1 (BHT-3021).

In some embodiments, the self-vector is administered in a pharmaceutically acceptable carrier or excipient.

In some embodiments, the self-vector is administered in a pharmaceutically acceptable carrier at a concentration about equal to physiological levels (e.g., about 0.9 mM).

In some embodiments, the self-vector is administered with a divalent cation at a concentration greater than physiological levels. In some embodiments, the divalent cation is calcium. In some embodiments, the calcium is at a concentration greater than about 2 mM; in some embodiments, the calcium is at a concentration of about 5.4 mM. In some embodiments, the self-vector is endotoxin-free. In some embodiments, the self-vector is administered intramuscularly. In some embodiments, the subject has IDDM.

In other aspects of the invention, any of the regimens disclosed here can be supplemented with co-administration of a polypeptide antigen, as described below.

Other objects, features, and advantages of the present invention will be apparent to one of skill in the art from the following detailed description and figures.

### BRIEF DESCRIPTION OF THE DRAWINGS

FIG. 1: Structural Vector Diagram of BHT-3021. The self-vector BHT-3021, a BHT-1 vector backbone with a sequence encoding a proinsulin self-protein, is shown with its component parts labeled. A CMV promoter drives expression of human proinsulin. Bovine growth hormone termination and polyA sequences (bGH pA) are incorporated 3′ to human proinsuling. Vector propagation and selection is accomplished via pUC origin of replication and a Kanamycin resistance gene (Kanr), respectively. BHT-3021 is 3324 basepairs and the location of each component is specified to the left of the vector map.

FIG. 2: Treatment of Established Hyperglycemia With DNA Vaccination Using BHT-3021 Formulated With Different Ca++ Concentrations. Female NOD mice were treated with weekly intramuscular DNA vaccinations after the onset of hyperglycemia (190-250 mg/dl) at treatment week 0. Fifty g of each DNA plasmid was administered per animal. The DNA vaccine BHT-3021 was injected at different Ca++ concentrations including: 0.9 mM (1×), 2.7 mM (3×) and 5.4 mM (6×). Animals were monitored weekly for IDDM onset and were considered diabetic on the first of 2 consecutive weeks with blood glucose levels greater than 250 mg/dl. Shown are the percentages of diabetic animals treated over time. KM plots were generated using GraphPad Prism. A) Treatment with BHT-3021 in different calcium concentrations without bupivacaine (tradename=Markane) revealed that a 6× calcium formulation significantly increased the efficacy of DNA vaccination to protect against progression to diabetes. B) Treatment with BHT-3021 at different calcium concentrations in combination with co-administration of insulin similarly revealed an increased efficacy against diabetes progression for a formulation utilizing 6× calcium. C) Treatment with BHT-3021 at different calcium concentrations and with bupivacaine revealed a slight increase in efficacy at 3× and 6× calcium formulations. D) Summary of results from experiments with BHT-3021 self-vector formulated with increasing concentrations of calcium with and without bupivacaine. E) Treatment of post-diabetic animals with BHT-3021 formulated with 1× or 6× calcium revealed a delay and reduction in the percentage of animals with high blood glucose levels with 6× calcium (lower right graph, dashed line with triangles), similar to anti-CD3 treated positive controls (left graph), compared to 1× calcium (upper right graph, dashed line with diamonds) that showed no difference from PBS treated controls (solid lines with squares). F) Treatment of post-diabetic animals with BHT-3021 formulated with 6× calcium (lower right graph, dashed line with triangles) or formulated with 1× calcium injected for 5 days (left graph, dashed line with open circles) reduced blood glucose levels compared to PBS treated controls (solid line with squares), an effect not seen with 1× calcium formulation alone (upper right graph, dashed line with diamonds). G) One-fifth of animals treated with BHT-3021 self-vector formulated with 6× calcium or formulated with 1× calcium injected for 5 days reverted to non-diabetic status as compared to no reversion in animals treated with 1× calcium or PBS.

FIG. 3: Reduction in antibodies to insulin in patients treated with a proinsulin encoding DNA plasmid vector. In a phase 1/2 trial, type 1 diabetic patients who were positive for anti-insulin antibodies at baseline (week 0) were treated with 12 weekly intramuscular 1 mg injections of a proinsulin encoding DNA plasmid vector (BHT-3021) constructed from the pBHT1 plasmid backbone. Antibody titers to three pancreatic autoantigens were measured at weeks 0, 2, 4, 6, 8, and 15 where available. The three antibodies, measured by radioimmunoassay and expressed as radioactivity index units, are antibodies to GAD, ICA512, and insulin (mIAA). In panel A is a patient treated with placebo (saline) injections who had positive antibody titers to GAD and insulin at baseline, but whose antibody titers did not change with treatment. In panel B is a patient treated with BHT-3021 who had positive antibody titers to GAD and insulin at baseline, and whose antibody titers to insulin decreased with treatment. In panel C is a patient treated with BHT-3021 who had positive antibody titers to ICA512 and insulin at baseline, and whose antibody titers to insulin decreased with treatment. These data demonstrate that BHT-3021 causes antigen-specific immune tolerance as demonstrated by rapid and sustained reductions in anti-insulin titers.

FIG. 4: Preservation of C-peptide in human patients treated with a proinsulin encoding DNA plasmid vector. As a measure of residual pancreatic β cell function, blood C-peptide levels were measured in these same patients at baseline (BL), week 5, week 15, and month 6, where available. In panel A is the patient treated with placebo whose C-peptide level steadily declines with no treatment. In panel B are the two patients treated with BHT-3021 whose C-peptide levels either show a less rapid decline or a slight increase in value, thus indicating preservation of β cell function.

FIG. 5 illustrates the preservation of C-peptide in human patients receiving anti-CD3 antibody (according to the protocol published in Herold, et al, Diabetes (2005) 54:1763-1769) or different doses of BHT-3021. Weekly administration of 1 mg, 3 mg and 6 mg doses of BHT 3021 over a period of 12 weeks demonstrated comparable C-peptide preservation at 6 months in comparison to the non-specific therapy of administration of anti-CD3 antibody.

FIG. 6 illustrates mean C-peptide levels in human patients receiving different weekly doses of BHT-3021 over a period of 6 months. Whereas C-peptide levels decreased in patients receiving the BHT-placebo, patients receiving weekly administration of 1 mg, 3 mg and 6 mg doses of BHT 3021 over a period of 12 weeks had stabilized or increased mean C-peptide levels measured at 6 months after the first administration.

FIG. 7 demonstrates the preservation of C-peptide in human patients receiving anti-CD3 antibody (according to the protocol published in Herold, et al, New England J Med (2002) 346:1692-1698) or 1 mg doses of BHT-3021. Weekly administration of 1 mg doses of BHT 3021 over a period of 12 weeks demonstrated comparable mean C-peptide levels at 6 and 12 months in comparison to the non-specific therapy of administration of anti-CD3 antibody.

FIG. 8 illustrates the mean changes in C-peptide levels over a period of 12 months in patients receiving weekly administration of 1 mg doses of BHT 3021 over a period of 12 weeks.

FIG. 9 illustrates the mean changes in C-peptide and glycosylated hemoglobin HbA1c levels over a period of 12 months in patients receiving weekly administration of 1 mg doses of BHT 3021 over a period of 12 weeks.

FIG. 10 demonstrates changes in glycosylated hemoglobin HbA1c levels over a period of 6 and 12 months in patients receiving weekly administration of 0.3 mg, 1 mg, 3 mg or 6 mg doses of BHT 3021 over a period of 12 weeks. HbA1c is lower in blood from patients treated with BHT-3021 in comparison to blood from patients receiving the placebo.

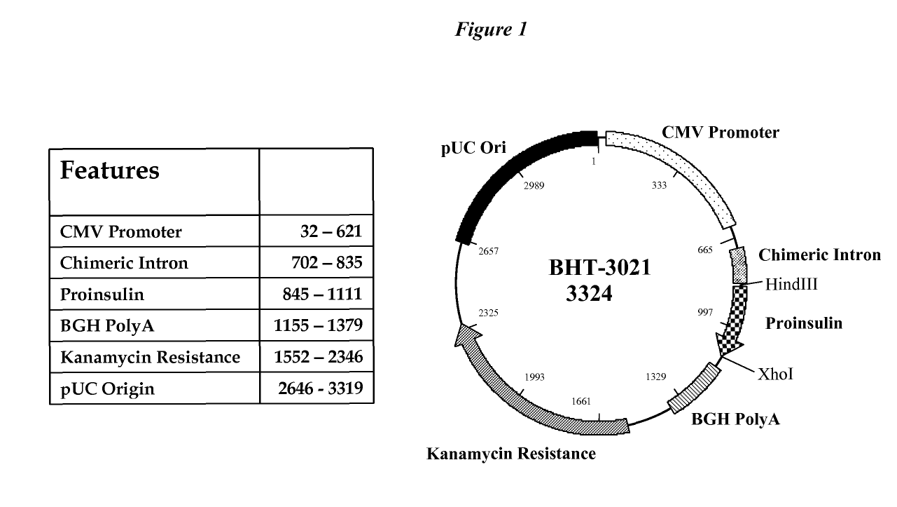

### DETAILED DESCRIPTION OF THE INVENTION

### I. Introduction

The present invention provides compositions and methods of treating, reducing, preventing, and inhibiting insulin-dependent diabetes mellitus (IDDM) by administration of a self-vector encoding and capable of expressing human proinsulin. It has surprisingly been found that frequent dosing (i.e., weekly or bi-weekly) of a low dose (i.e., 1 to 3 mg per administration) of a DNA self-vector in a subject suffering from IDDM is efficacious in reducing the severity of disease. No stimulatory immune response against the autoantigen expressed by the self-vector is induced. Moreover, administration of higher doses are not more efficacious in reducing severity of disease.

As described above, in IDDM, prior to the onset of overt diabetes, there is a long presymptomatic period during which there is a gradual loss of pancreatic β cell function. Markers that can be evaluated include without limitation blood or serum levels of C-peptide as indicative of pancreatic β cell function, the presence of insulitis in the pancreas, the level and frequency of islet cell antibodies, islet cell surface antibodies, the presence and concentration of autoantibodies against autoantigens or self-protein targeted in IDDM, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration.

The Non-Obese Diabetic (NOD) mouse is an animal model with many clinical, immunological, and histopathological features in common with human IDDM. NOD mice spontaneously develop inflammation of the islets and destruction of the β cells, which leads to hyperglycemia and overt diabetes. Both CD4⁺ and CD8⁺ T cells are required for diabetes to develop, although the roles of each remain unclear. It has been shown that administration of insulin or GAD, as proteins, under tolerizing conditions to NOD mice prevents disease and down-regulates responses to the other autoantigens.

The presence of combinations of autoantibodies with various specificities in serum are highly sensitive and specific for human type I diabetes mellitus. For example, the presence of autoantibodies against GAD and/or IA-2 is approximately 98% sensitive and 99% specific for identifying type I diabetes mellitus from control serum. In non-diabetic first degree relatives of type I diabetes patients, the presence of autoantibodies specific for two of the three autoantigens or self-proteins, including GAD, insulin and IA-2 conveys a positive predictive value of >90% for development of type IDM within 5 years.

Autoantigens or self-proteins targeted in human insulin dependent diabetes mellitus include, for example, insulin autoantigens, including insulin, insulin B chain, proinsulin, and preproinsulin; tyrosine phosphatase IA-2; IA-2β; glutamic acid decarboxylase (GAD) both the 65 kDa and 67 kDa forms; carboxypeptidase H; heat shock proteins (HSP); glima 38; islet cell antigen 69 KDa (ICA69); p52; two ganglioside antigens (GT3 and GM2-1); islet-specific glucose-6-phosphatase-related protein (IGRP); zinc transporter Slc30A8; and an islet cell glucose transporter (GLUT 2).

Accordingly, the present invention provides compositions and methods for treating, preventing, reducing, inhibiting and/or delaying, e.g., the symptoms of or the severity of IDDM in a subject comprising administration of a modified self-vector encoding and capable of expressing human proinsulin, in particular, the self-vector BHT-3021 (SEQ ID NO: 1). Administration of a therapeutically or prophylactically effective amount of the modified self-vector to a subject elicits suppression of an immune response against an autoantigen or self-protein associated with or targeted in IDDM, thereby treating or preventing the disease. The self-vector may be co-administered or co-formulated with one or more divalent cations present at higher than physiologic concentrations. Surprisingly, co-administration of the self-vector with one or more divalent cations at total concentration higher than physiologic levels improves one or more of transfection efficiency, expression (i.e., transcription and translation) of the encoded autoantigen, and therapeutic suppression of an undesirable immune response in comparison to co-administration of a self-vector in the presence of one or more divalent cations at total concentration equal to or lower than physiologic levels.

### II. Definitions

Unless defined otherwise, all technical and scientific terms used herein have the same meaning as commonly understood by those of ordinary skill in the art to which this invention belongs. The following references provide one of skill with a general definition of many of the terms used herein: Hale and Margham, The Harper Collins Dictionary of Biology (HarperPerennial, 1991); King and Stansfield, A Dictionary of Genetics (Oxford University Press, 4th ed. 1990); Stedman's Medical Dictionary (Lippincott Williams & Wilkins, 27th ed. 2000); and Hawley's Condensed Chemical Dictionary (John Wiley & Sons, 13th ed. 1997). As used herein, the following terms and phrases have the meanings ascribed to them unless specified otherwise.

The terms “a,” “an,” and “the” include plural referents unless the context clearly dictates otherwise.

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, DNA, synthetic forms, and mixed polymers, both sense and antisense strands, double- or single-stranded, and can also be chemically or biochemically modified or can contain non-natural or derivatized nucleotide bases, as will be readily appreciated by the skilled artisan. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The terms “intron” or “intronic sequence” as used herein refers to intervening polynucleotide sequences within a gene or portion of a gene present in a self-vector that is situated upstream of or between “exons”, polynucleotide sequences that are retained during RNA processing and most often code for a polypeptide. Introns do not function in coding for protein synthesis and are spliced out of a RNA before it is translated into a polypeptide.

“Splicing” refers to the mechanism by which a single functional RNA molecule is generated by the removal of introns and juxtaposition of exons during processing of the primary transcript, or preRNA. Consensus sequences are present at intron-exon junctions that define the 5′ end, or donor site, of an intron and the 3′ end, or acceptor site, and at a branchpoint site located approximately 20-50 basepairs upstream of the acceptor site within the intron sequence. Most introns start from the sequence GU and end with the sequence AG (in the 5′ to 3′ direction) with a branchpoint site approximating CU(A/G)A(C/U), where A is conserved in all genes. These sequences signal for the looping out of the intron and its subsequent removal.

The term “promoter” is used here to refer to the polynucleotide region recognized by RNA polymerases for the initiation of RNA synthesis, or “transcription”. Promoters are one of the functional elements of self-vectors that regulate the efficiency of transcription and thus the level of protein expression of a self-polypeptide encoded by a self-vector. Promoters can be “constitutive”, allowing for continual transcription of the associated gene, or “inducible”, and thus regulated by the presence or absence of different substances in the environment. Additionally, promoters can also either be general, for expression in a broad range of different cell types, or cell-type specific, and thus only active or inducible in a particular cell type, such as a muscle cell. Promoters controlling transcription from vectors may be obtained from various sources, for example, the genomes of viruses such as: polyoma, simian virus 40 (SV40), adenovirus, retroviruses, hepatitis B virus and cytomegalovirus, or from heterologous mammalian promoters, e.g., β-actin promoter. The early and late promoters of the SV40 virus are conveniently obtained as is the immediate early promoter of the human cytomegalovirus.

“Enhancer” refers to cis-acting polynucleotide regions of about from 10-300 basepairs that act on a promoter to enhance transcription from that promoter. Enhancers are relatively orientation and position independent and can be placed 5′ or 3′ to the transcription unit, within introns, or within the coding sequence itself.

A “terminator sequence” as used herein means a polynucleotide sequence that signals the end of DNA transcription to the RNA polymerase. Often the 3′ end of a RNA generated by the terminator sequence is then processed considerably upstream by polyadenylation. “Polyadenylation” is used to refer to the non-templated addition of about 50 to about 200 nucleotide chain of polyadenylic acid (polyA) to the 3′ end of a transcribed messenger RNA. The “polyadenylation signal” (AAUAAA) is found within the 3′ untranslated region (UTR) of a mRNA and specifies the site for cleavage of the transcript and addition of the polyA tail. Transcription termination and polyadenylation are functionally linked and sequences required for efficient cleavage/polyadenylation also constitute important elements of termination sequences (Connelly and Manley, 1988).

“Oligonucleotide,” as used herein refers, to a subset of polynucleotides of from about 6 to about 175 nucleotides or more in length. Typical oligonucleotides are up to about 100 nucleotides in length. Oligonucleotide refers to both oligoribonucleotides and to oligodeoxyribonucleotides, hereinafter referred to ODNs. ODNs include oligonucleosides and other organic base containing polymers. Oligonucleotides can be obtained from existing nucleic acid sources, including genomic DNA, plasmid DNA, viral DNA and cDNA, but are typically synthetic oligonucleotides produced by oligonucleotide synthesis. Oligonucleotides can be synthesized on an automated oligonucleotide synthesizer (for example, those manufactured by Applied BioSystems (Foster City, Calif.)) according to specifications provided by the manufacturer.

The terms “DNA vaccination”, “DNA immunization”, and “polynucleotide therapy” are used interchangeably herein and refer to the administration of a polynucleotide to a subject for the purpose of modulating an immune response. “DNA vaccination” with plasmids expressing foreign microbial antigens is a well known method to induce protective antiviral or antibacterial immunity (Davis, 1997; Hassett and Whitton, 1996; and Ulmer et al., 1996). For the purpose of the present invention, “DNA vaccination”, “DNA immunization”, or “polynucleotide therapy” refers to the administration of polynucleotides encoding one or more self-polypeptides that include one or more autoantigenic epitopes associated with or targeted in an autoimmune disease. The “DNA vaccination” serves the purpose of modulating an ongoing immune response to suppress autoimmune destruction for the treatment or prevention of an autoimmune disease. Modulation of an immune response in reaction to “DNA vaccination” may include shifting self-reactive lymphocytes from a Th1- to a Th2-type response. The modulation of the immune response may occur systemically or only locally at the target organ under autoimmune attack.

“Self-vector” (also referred to as a DNA plasmid vector) means one or more vector(s) which taken together comprise a polynucleotide either DNA or RNA encoding one or more self-protein(s), -polypeptide(s), -peptide(s), e.g., one or more autoantigens. Polynucleotide, as used herein is a series of either deoxyribonucleic acids including DNA or ribonucleic acids including RNA, and their derivatives, encoding a self-protein, -polypeptide, or -peptide of this invention. The self-protein, -polypeptide or -peptide coding sequence is inserted into an appropriate plasmid expression self-cassette. Once the polynucleotide encoding the self-protein, -polypeptide, or -peptide is inserted into the expression self-cassette the vector is then referred to as a “self-vector.” In the case where polynucleotide encoding more than one self-protein(s), -polypeptide(s), or -peptide(s) is to be administered, a single self-vector may encode multiple separate self-protein(s), -polypeptide(s) or -peptide(s). In one embodiment, DNA encoding several self-protein(s), -polypeptide(s), or -peptide(s) are encoded sequentially in a single self-plasmid utilizing internal ribosomal re-entry sequences (IRES) or other methods to express multiple proteins from a single DNA molecule. The DNA expression self-vectors encoding the self-protein(s), -polypeptide(s), or -peptide(s) are prepared and isolated using commonly available techniques for isolation of plasmid DNA such as those commercially available from Qiagen Corporation. The DNA is purified free of bacterial endotoxin for delivery to humans as a therapeutic agent. Alternatively, each self-protein, -polypeptide or -peptide is encoded on a separate DNA expression vector. In some embodiments, the self-vector is a DNA plasmid vector.

The term “vector backbone” refers to the portion of a plasmid vector other than the sequence encoding a self-antigen, -protein, -polypeptide, or -peptide.

“Plasmids” and “vectors” are designated by a lower case p followed by letters and/or numbers. The starting plasmids are commercially available, publicly available on an unrestricted basis, or can be constructed from available plasmids in accord with published procedures. In addition, equivalent plasmids to those described are known in the art and will be apparent to the ordinarily skilled artisan. A “vector” or “plasmid” refers to any genetic element that is capable of replication by comprising proper control and regulatory elements when present in a host cell. For purposes of this invention examples of vectors or plasmids include, but are not limited to, plasmids, phage, transposons, cosmids, virus, and the like.

“Transfection” means introducing DNA into a host cell so that the DNA is expressed, whether functionally expressed or otherwise; the DNA may also replicate either as an extrachromosomal element or by chromosomal integration. Transfection may be accomplished by any method known in the art suitable for introducing an extracellular nucleic acid into a host cell, including but not limited to, the use of transfection facilitating agents or processes such as calcium phosphate co-precipitation, viral transduction, protoplast fusion, DEAE-dextran-mediated transfection, polybrene-mediated transfection, liposome fusion, microinjection, microparticle bombardment or electroporation. In some embodiments, the nucleic acid of interest is formulated with calcium for injection into an animal for uptake by the host cells of the animal. In some embodiments, the nucleic acid to be transfected is formulated with calcium at a concentration between about 0.05 mM to about 2 M; in some embodiments the calcium concentration is between about 0.9 mM (1×) to about 8.1 mM (9×); in some embodiments the calcium concentration is between about 0.9 mM (1×) to about 5.4 mM (6×).

“Antigen,” as used herein, refers to any molecule that can be recognized by the immune system that is by B cells or T cells, or both.

“Autoantigen,” as used herein, refers to an endogenous molecule, typically a protein or fragment thereof, that elicits a pathogenic immune response. When referring to the autoantigen or epitope thereof as “associated with an autoimmune disease,” it is understood to mean that the autoantigen or epitope is involved in the pathophysiology of the disease either by inducing the pathophysiology (i.e., associated with the etiology of the disease), mediating or facilitating a pathophysiologic process; and/or by being the target of a pathophysiologic process. For example, in autoimmune disease, the immune system aberrantly targets autoantigens, causing damage and dysfunction of cells and tissues in which the autoantigen is expressed and/or present. Under normal physiological conditions, autoantigens are ignored by the host immune system through the elimination, inactivation, or lack of activation of immune cells that have the capacity to recognize the autoantigen through a process designated “immune tolerance.”

Autoantigens targeted in human insulin dependent diabetes mellitus may include, for example, tyrosine phosphatase IA-2; IA-2P; glutamic acid decarboxylase (GAD) both the 65 kDa and 67 kDa forms; carboxypeptidase H; insulin; proinsulin (e.g., SEQ ID NOs: 1 and 2); heat shock proteins (HSP); glima 38; islet cell antigen 69 KDa (ICA69); p52; two ganglioside antigens (GT3 and GM2-1); islet-specific glucose-6-phosphatase-related protein (IGRP); zinc transporter Slc30A8, and an islet cell glucose transporter (GLUT 2).

As used herein the term “epitope” is understood to mean a portion of a polypeptide having a particular shape or structure that is recognized by either B-cells or T-cells of the animal's immune system. “Autoantigenic epitope” or “pathogenic epitope” refers to an epitope of an autoantigen that elicits a pathogenic immune response. The immunodominant epitopes of autoantigens targeted in IDDM and/or related diseases are known in the art. See, e.g., Hawkes, et al., Diabetes (2000) 49(3):356-366 (IA-2); Gebe, et al., Clinical Immunol (2006) 121(3):294-304 (GAD); Lich, et al., J Immunol (2003): 171: 853-859 (GAD); Falorni, et al., Diabetologia (1996) 39(9):1091-98 (GAD); Patel, et al, PNAS (1997) 94(15):8082-8087 (GAD); Congia, et al., PNAS (1998) 95(7):3833-3838 (insulin); Higashide, et al., Pediatr Res. (2006) 59(3):445-50 (insulin); Marttila, et al., J Autoimmun. (2008) 31(2):142-8 (insulin); Polanski, et al., J Autoimmun. (1997) 10(4):339-46 (insulin); Panagiotopoulos, et al., Curr Diab Rep. (2004) 4(2):87-94 (review); and Descamps, et al., Adv Exp Med Biol. (2003) 535:69-77 (review).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Self-protein,” “self-polypeptide,” self-peptide,” or “autoantigen” are used herein interchangeably and refer to any protein, polypeptide, or peptide, or fragment or derivative thereof that: is encoded within the genome of the animal; is produced or generated in the animal; may be modified posttranslationally at some time during the life of the animal; and, is present in the animal non-physiologically. The term “non-physiological” or “non-physiologically” when used to describe the self-protein(s), -polypeptide(s), or -peptide(s) of this invention means a departure or deviation from the normal role or process in the animal for that self-protein, -polypeptide, or -peptide. When referring to the self-protein, -polypeptide or -peptide as “associated with a disease,” “targeted in a disease” or “involved in a disease” it is understood to mean that the self-protein, -polypeptide, or -peptide may be modified in form or structure and thus be unable to perform its physiological role or process or may be involved in the pathophysiology of the condition or disease either by inducing the pathophysiology; mediating or facilitating a pathophysiologic process; and/or by being the target of a pathophysiologic process. For example, in autoimmune disease, the immune system aberrantly attacks self-proteins causing damage and dysfunction of cells and tissues in which the self-protein is expressed and/or present. Alternatively, the self-protein, -polypeptide or -peptide can itself be expressed at non-physiological levels and/or function non-physiologically. For example in neurodegenerative diseases self-proteins are aberrantly expressed, and aggregate in lesions in the brain thereby causing neural dysfunction. In other cases, the self-protein aggravates an undesired condition or process. For example in osteoarthritis, self-proteins including collagenases and matrix metalloproteinases aberrantly degrade cartilage covering the articular surface of joints. Examples of posttranslational modifications of self-protein(s), -polypeptide(s) or -peptide(s) are glycosylation, addition of lipid groups, reversible phosphorylation, addition of dimethylarginine residues, citrullination, and proteolysis, and more specifically citrullination of fillagrin and fibrin by peptidyl arginine deiminase (PAD), alpha β-crystallin phosphorylation, citrullination of MBP, and SLE autoantigen proteolysis by caspases and granzymes. Immunologically, self-protein, -polypeptide or -peptide would all be considered host self-antigens and under normal physiological conditions are ignored by the host immune system through the elimination, inactivation, or lack of activation of immune cells that have the capacity to recognize self-antigens through a process designated “immune tolerance.”

A self-protein, -polypeptide, or -peptide does not include immune proteins, polypeptides, or peptides which are molecules expressed physiologically exclusively by cells of the immune system for the purpose of regulating immune function. The immune system is the defense mechanism that provides the means to make rapid, highly specific, and protective responses against the myriad of potentially pathogenic microorganisms inhabiting the animal's world. Examples of immune protein(s), polypeptide(s) or peptide(s) are proteins comprising the T-cell receptor, immunoglobulins, cytokines including the type I interleukins, and the type II cytokines, including the interferons and IL-10, TNF, lymphotoxin, and the chemokines such as macrophage inflammatory protein-1 alpha and beta, monocyte-chemotactic protein and RANTES, and other molecules directly involved in immune function such as Fas-ligand. There are certain immune protein(s), polypeptide(s) or peptide(s) that are included in the self-protein, -polypeptide or -peptide of the invention and they are: class I MHC membrane glycoproteins, class II MHC glycoproteins and osteopontin. Self-protein, -polypeptide or -peptide does not include proteins, polypeptides, and peptides that are absent from the subject, either entirely or substantially, due to a genetic or acquired deficiency causing a metabolic or functional disorder, and are replaced either by administration of said protein, polypeptide, or peptide or by administration of a polynucleotide encoding said protein, polypeptide or peptide (gene therapy). Examples of such disorders include Duchenne' muscular dystrophy, Becker's muscular dystrophy, cystic fibrosis, phenylketonuria, galactosemia, maple syrup urine disease, and homocystinuria. Self-protein, -polypeptide or -peptide does not include proteins, polypeptides, and peptides expressed specifically and exclusively by cells which have characteristics that distinguish them from their normal counterparts, including: (1) clonality, representing proliferation of a single cell with a genetic alteration to form a clone of malignant cells, (2) autonomy, indicating that growth is not properly regulated, and (3) anaplasia, or the lack of normal coordinated cell differentiation. Cells have one or more of the foregoing three criteria are referred to either as neoplastic, cancer or malignant cells.

“Modulation of,” “modulating”, or “altering an immune response” as used herein refers to any alteration of an existing or potential immune responses against self-molecules, including, e.g., nucleic acids, lipids, phospholipids, carbohydrates, self-polypeptides, protein complexes, or ribonucleoprotein complexes, that occurs as a result of administration of a polynucleotide encoding a self-polypeptide. Such modulation includes any alteration in presence, capacity, or function of any immune cell involved in or capable of being involved in an immune response. Immune cells include B cells, T cells, NK cells, NK T cells, professional antigen-presenting cells, non-professional antigen-presenting cells, inflammatory cells, or any other cell capable of being involved in or influencing an immune response. “Modulation” includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response.

“Modulation of an immune response” includes, for example, the following: elimination, deletion, or sequestration of immune cells; induction or generation of immune cells that can modulate the functional capacity of other cells such as autoreactive lymphocytes, antigen presenting cells (APCs), or inflamatory cells; induction of an unresponsive state in immune cells (i.e., anergy); increasing, decreasing, or changing the activity or function of immune cells or the capacity to do so, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors; or any combination of these modulatory events.

For example, a polynucleotide encoding a self-polypeptide can modulate an immune response by eliminating, sequestering, or inactivating immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of an immune response include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T cell proliferation assays and pepscan analysis based on ³H-thymidine incorporation following stimulation with anti-CD3 antibody, anti-T cell receptor antibody, anti-CD28 antibody, calcium ionophores, PMA, antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimentional gel electrophoresis, Western blot analysis, enzyme linked immunosorbent assays, flow cytometry); and measurement of clinical symptoms or outcomes such as improvement of autoimmune, neurodegenerative, and other diseases involving self proteins or self polypeptides (clinical scores, requirements for use of additional therapies, functional status, imaging studies) for example, by measuring relapse rate or disease severity (using clinical scores known to the ordinarily skilled artisan) in the case of multiple sclerosis, measuring blood glucose in the case of type I diabetes, or joint inflammation in the case of rheumatoid arthritis.

“Subjects” shall mean any animal, such as, for example, a human, non-human primate, horse, cow, dog, cat, mouse, rat, guinea pig or rabbit.

“Treating,” “treatment,” or “therapy” of a disease or disorder shall mean slowing, stopping or reversing the disease's progression, as evidenced by decreasing, cessation or elimination of either clinical or diagnostic symptoms, by administration of a polynucleotide encoding a self-polypeptide, either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. In some embodiments, treating a disease means reversing or stopping or mitigating the disease's progression, ideally to the point of eliminating the disease itself. As used herein, ameliorating a disease and treating a disease are equivalent.

“Preventing,” “prophylaxis,” or “prevention” of a disease or disorder as used in the context of this invention refers to the administration of a polynucleotide encoding a self-protein or autoantigen, either alone or in combination with another compound as described herein, to prevent the occurrence or onset of a disease or disorder or some or all of the symptoms of a disease or disorder or to lessen the likelihood of the onset of a disease or disorder.

“Insulin-dependent diabetes mellitus,” “human type I,” and “insulin-dependent diabetes mellitus and/or related diseases” refers to diseases characterized by the autoimmune destruction of the β cells in the pancreatic islets of Langerhans. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. Included within insulin-dependent diabetes mellitus and related diseases are asymptomatic diabetes (evidenced by antibodies to islet antigens), genetically pre-disposed diabetes, new onset or incident diabetes (for example, patients with greater than 0.033 nm/l C-peptide or such other level of C-peptide depending on assay sensitivity), prevalent diabetes, type I diabetes mellitus, individuals between 19 and 40 years of age within five (5) years of diagnosis (for example, patients with greater than 0.033 nm/l C-peptide or such other level depending on assay sensitivity), latent adult onset diabetes (LADA), islet transplantation to block the recurrence of autoimmune disease, type 2 diabetics who have evidence of autoimmunity (evidenced by antibodies to islet antigens) or in combination with therapeutic agents to stimulate islet regeneration.

The term “regimen” refers to a regulated set of parameters for treatment, prophylaxis and/or maintenance of an IDDM and/or related diseases, particularly with respect to configuring three parameters—dose, frequency of administration and the period of treatment. The three parameters comprising a regimen are: (1) a therapeutically effective dose or amount of the DNA self-vector or DNA plasmid; (2) the frequency of administration of the DNA self-vector (i.e., how frequently is each therapeutically effective dose of DNA self-vector or DNA plasmid given, e.g. weekly or bi-weekly); and (3) the time period over which the DNA self-vectors or plasmid (i.e., how long is the treatment administered, e.g. continuous dosing, four (4) weeks of dosing, six (6) weeks of dosing, twelve (12) weeks of dosing, one (1) year of dosing, eighteen (18) months of dosing or twenty-four (24) months of dosing.). A “regimen” can be for prevention, treatment, or maintenance of disease.

A “treatment regimen” or “therapeutic regimen” refers to regimen carried out on a patient for the purposes of treatment, as described above, e.g., for slowing, stopping or reversing the disease's progression, as evidenced by decreasing, cessation or elimination of either clinical or diagnostic symptoms. A treatment regimen is performed to reduce disease severity, improve and stabilize the disease symptoms of the patient.

A “supplemental regimen” or “maintenance regimen” is carried out on a patient whose disease symptoms are stabilized, e.g., by having previously received a therapeutic or treatment regimen.

A “therapeutically or prophylactically effective amount” of a self-vector refers to an amount of the self-vector that is administered at a particular frequency over a certain period as taught by the present invention sufficient to treat or prevent the disease as, for example, by ameliorating or eliminating symptoms and/or the cause of the disease. For example, therapeutically effective amounts fall within broad range(s) and are determined through clinical trials and for a particular patient is determined based upon factors known to the skilled clinician, including, e.g., the severity of the disease, weight of the patient, age, and other factors. Therapeutically effective amounts of self-vector are in the range of about 0.3 mg to about 6 mg. A preferred therapeutic amount of self-vector is in the range of about 1 mg to about 6 mg. A most preferred therapeutic amount of self-vector is in the range of about 1 mg to 3 mg, for example, 1 mg, 2 mg or 3 mg per administration.

The term “dosing frequency” or “frequency of dosing” refers to the time interval between administration of the DNA self-vector. The dosing frequency of the DNA self-vector can be daily, weekly, bi-weekly (i.e., once every other week or twice monthly), monthly, bi-monthly (i.e., once every other month), semi-annually (i.e., twice yearly) or annually. In preferred embodiments, the dosing frequency in a treatment regimen is weekly or bi-weekly.

The term “dosing period” or “time period of dosing” refers to the time period between the first and last administration of a therapeutically effective amount of DNA self-vector or DNA plasmid that is administered at a certain frequency.

The term “continuous” refers to a time period of dosing that is uninterrupted or without a break such as for the life of the patient or until a desired therapeutic endpoint is reached.

The term “route of administration” refers to the path by which a DNA self-vector or plasmid is brought into contact with the patient. The route of administration may be i.v., parenteral, sub-cutaneous, or intramuscular. In one aspect intramuscular administration is carried out by injecting the DNA self-vector or plasmid in one, two, three or more sites in the subject's body.

The term “co-administration” refers to the presence of the two or more active agents (e.g., a self-vector and a polypeptide autoantigen) in the blood at the same time. Co-administration can be concurrent or sequential. The co-administered active agents can be administered together or separately.

The phrase “endotoxin-free” refers to a vector or a composition of the invention that is substantially free of endotoxin, e.g., has endotoxin contamination below detectable levels. A vector or composition that is endotoxin-free can be described in terms of a threshold concentration of detectable endotoxin as measured using a Limulus Amebocyte Lysate (LAL) gel clot assay, known in the art. With respect to a threshold concentration, a vector or composition is endotoxin-free if the amount of contaminating endotoxin is below the limit of detection (e.g., less than about 0.10 endotoxin units/ml or EU/ml). To the extent that endotoxin can be detected, a vector or composition is substantially endotoxin-free if the amount of contaminating endotoxin is below about 2.5 EU/ml. Numerous companies provide commercially available testing services to determine the level of endotoxin in a preparation, including e.g., Nelson Laboratories, Salt Lake City, Utah; Boehringer Ingelheim, Austria; MO BIO Laboratories, Carlsbad, Calif.; NovaTX, Conroe, Tex.; and Associates of Cape Cod, Inc., East Falmouth, Mass. LAL gel clot detection kits are also available for purchase, from for example, Lonza, on the worldwide web at lonza.com and Charles River Laboratories, on the worldwide web at criver.com.

### III. Descriptions of the Embodiments

A. BHT-3021 Self-Vector

In some embodiments, the present invention provides a self-vector or DNA plasmid vector of SEQ ID NO:1 (BHT-3021). The self-vector BHT-3021 comprises a BHT-1 expression vector backbone and a polynucleotide encoding human proinsulin. The self-vector BHT-3021 also comprises a CMV promoter, which drives the expression of human proinsulin; bovine growth hormone termination and polyA sequences; and a pUC origin of replication and a Kanamycin resistance gene (Kanr), which accomplish vector propagation and selection, respectively.

The backbone of the BHT-3021 vector is a modified pVAX1 vector in which one or more CpG dinucleotides of the formula 5′-purine-pyrimidine-C-G-pyrimidine-pyrimidine-3′ is mutated by substituting the cytosine of the CpG dinucleotide with a non-cytosine nucleotide. The pVAX1 vector is known in the art and is commercially available from Invitrogen (Carlsbad, Calif.). In one exemplary embodiment, the modified pVAX1 vector has the following cytosine to non-cytosine substitutions within a CpG motif: cytosine to guanine at nucleotides 784, 1161, 1218, and 1966; cytosine to adenine at nucleotides 1264, 1337, 1829, 1874, 1940, and 1997; and cytosine to thymine at nucleotides 1158, 1963 and 1987; with additional cytosine to guanine mutations at nucleotides 1831, 1876, 1942, and 1999. (The nucleotide number designations as set forth above are according to the numbering system for pVAX1 provided by Invitrogen.)

The invention contemplates BHT-3021 vectors with added, deleted or substituted nucleotides that do not change the function of the BHT-3021 vector, e.g., for expressing proinsulin and inhibiting an autoimmune response. Accordingly, the invention contemplates a self-vector comprising a polynucleotide encoding human proinsulin that shares at least about 90%, 91%, 92%, 93%, 94%, 95%, 95%, 97%, 98% or 99% nucleic acid sequence identity to SEQ ID NO:1, as measured using an algorithm known in the art, e.g., BLAST or ALIGN, set with standard parameters. Sequence identity can be determined with respect to, e.g., the full-length of the BHT backbone, the full-length of the proinsulin autoantigen, or the full-length of the BHT-3021 vector.

Techniques for construction of vectors and transfection of cells are well-known in the art, and the skilled artisan will be familiar with the standard resource materials that describe specific conditions and procedures. The self-vector BHT-3021 is prepared and isolated using commonly available techniques for isolation of nucleic acids. The vector is purified free of bacterial endotoxin for delivery to humans as a therapeutic agent.

Construction of the vectors of the invention employs standard ligation and restriction techniques that are well-known in the art (see generally, e.g., Ausubel et al., Current Protocols in Molecular Biology, 1990-2008, John Wiley Interscience; Sambrook and Russell, Molecular Cloning: A Laboratory Manual, 2001, Cold Spring Harbor Laboratory Press). Isolated plasmids, DNA sequences, or synthesized oligonucleotides are cleaved, tailored, and relegated in the form desired. Sequences of DNA constructs can be confirmed using, e.g., standard methods for DNA sequence analysis (see, e.g., Sanger et al. (1977) Proc. Natl. Acad. Sci., 74, 5463-5467).

Nucleotide sequences selected for use in the self-vector can be derived from known sources, for example, by isolating the nucleic acid from cells containing a desired gene or nucleotide sequence using standard techniques. Similarly, the nucleotide sequences can be generated synthetically using standard modes of polynucleotide synthesis that are well known in the art. See, e.g., Edge et al., Nature 292:756, 1981; Nambair et al., Science 223:1299, 1984; Jay et al., J. Biol. Chem. 259:6311, 1984. Generally, synthetic oligonucleotides can be prepared by either the phosphotriester method as described by Edge et al. (supra) and Duckworth et al. (Nucleic Acids Res. 9:1691, 1981); or the phosphoramidite method as described by Beaucage et al. (Tet. Letts. 22:1859, 1981) and Matteucci et al. (J. Am. Chem. Soc. 103:3185, 1981). Synthetic oligonucleotides can also be prepared using commercially available automated oligonucleotide synthesizers. The nucleotide sequences can thus be designed with appropriate codons for a particular amino acid sequence. In general, one will select preferred codons for expression in the intended host. The complete sequence is assembled from overlapping oligonucleotides prepared by standard methods and assembled into a complete coding sequence. See, e.g., Edge et al. (supra); Nambair et al. (supra) and Jay et al. (supra).

Another method for obtaining nucleic acid sequences for use herein is by recombinant means. Thus, a desired nucleotide sequence can be excised from a plasmid carrying the nucleic acid using standard restriction enzymes and procedures. Site specific DNA cleavage is performed by treating with the suitable restriction enzymes and procedures. Site specific DNA cleavage is performed under conditions which are generally understood in the art, and the particulars of which are specified by manufacturers of commercially available restriction enzymes. If desired, size separation of the cleaved fragments may be performed by polyacrylamide gel or agarose gel electrophoreses using standard techniques.

Yet another convenient method for isolating specific nucleic acid molecules is by the polymerase chain reaction (PCR) (Mullis et al., Methods Enzymol. 155:335-350, 1987) or reverse transcription PCR (RT-PCR). Specific nucleic acid sequences can be isolated from RNA by RT-PCR. RNA is isolated from, for example, cells, tissues, or whole organisms by techniques known to one skilled in the art. Complementary DNA (cDNA) is then generated using poly-dT or random hexamer primers, deoxynucleotides, and a suitable reverse transcriptase enzyme. The desired polynucleotide can then be amplified from the generated cDNA by PCR. Alternatively, the polynucleotide of interest can be directly amplified from an appropriate cDNA library. Primers that hybridize with both the 5′ and 3′ ends of the polynucleotide sequence of interest are synthesized and used for the PCR. The primers may also contain specific restriction enzyme sites at the 5′ end for easy digestion and ligation of amplified sequence into a similarly restriction digested plasmid vector.

The expression cassette of the modified self-vector will employ a promoter that is functional in host cells. In general, vectors containing promoters and control sequences that are derived from species compatible with the host cell are used with the particular host cell. Promoters suitable for use with prokaryotic hosts illustratively include the beta-lactamase and lactose promoter systems, alkaline phosphatase, the tryptophan (trp) promoter system and hybrid promoters such as tac promoter. However, other functional bacterial promoters are suitable. In addition to prokaryotes, eukaryotic microbes such as yeast cultures may also be used. Saccharomyces cerevisiae, or common baker's yeast is the most commonly used eukaryotic microorganism, although a number of other strains are commonly available. Promoters controlling transcription from vectors in mammalian host cells may be obtained from various sources, for example, the genomes of viruses such as: polyoma, simian virus 40 (SV40), adenovirus, retroviruses, hepatitis B virus and cytomegalovirus (CMV), or from heterologous mammalian promoters, e.g. β-actin promoter. The early and late promoters of the SV 40 virus are conveniently obtained as an SV40 restriction fragment which also contains the SV40 viral origin of replication. The immediate early promoter of the human cytomegalovirus is conveniently obtained as a HindIII restriction fragment. Of course, promoters from the host cell or related species also are useful herein.

For in vitro evaluation, host cells may be transformed with the modified self-vector and cultured in conventional nutrient media modified as is appropriate for inducing promoters, selecting transformants or amplifying genes. One suitable method for transfection of the host cells is the calcium phosphate co-precipitation method of Graham and van der Eb (1973) Virology 52, 456-457. Alternative methods for transfection are electroporation, the DEAE-dextran method, lipofection and biolistics (Kriegler (1990) Gene Transfer and Expression: A Laboratory Manual, Stockton Press). Culture conditions, such as temperature, pH and the like, that are suitable for host cell expression are generally known in the art and will be apparent to the skilled artisan.

Modified self-vectors of this invention can be formulated as polynucleotide salts for use as pharmaceuticals. Polynucleotide salts can be prepared with non-toxic inorganic or organic bases. Inorganic base salts include sodium, potassium, zinc, calcium, aluminum, magnesium, etc. Organic non-toxic bases include salts of primary, secondary and tertiary amines, etc. Such self-DNA polynucleotide salts can be formulated in lyophilized form for reconstitution prior to delivery, such as sterile water or a salt solution. Alternatively, self-DNA polynucleotide salts can be formulated in solutions, suspensions, or emulsions involving water- or oil-based vehicles for delivery. In one embodiment, the DNA is lyophilized in phosphate buffered saline with physiologic levels of calcium (0.9 mM) and then reconstituted with sterile water prior to administration. Alternatively the DNA is formulated in solutions containing higher quantities of Ca⁺⁺, between 1 mM and 2M. The DNA can also be formulated in the absence of specific ion species.

B. Compositions

In some embodiments, the present invention provides a composition comprising a self-vector or DNA plasmid vector of SEQ ID NO:1 (BHT-3021). The composition can be formulated in a pharmaceutically acceptable carrier. In some embodiments, the pharmaceutical composition comprises calcium at a concentration about equal to physiological levels (e.g., about 0.9 mM). In some embodiments, the pharmaceutical composition further comprises a divalent cation at a concentration greater than physiological levels. In some embodiments, the divalent cation is calcium. In some embodiments, the self-vector is formulated with calcium at a concentration between about 0.9 mM (1×) to about 2 M; in some embodiments the calcium concentration is between about 2 mM to about 8.1 mM (9×); in some embodiments the calcium concentration is between about 2 mM to about 5.4 mM (6×). In some embodiments, the pharmaceutical composition is endotoxin-free.

In some embodiments, the self-vector is formulated with one or more divalent cations at a total concentration greater than physiological levels for injection into an animal for uptake by the host T cells of the animal. In some embodiments, one or more physiologically acceptable divalent cations can be used, e.g., Ca²⁺, Mg²⁺, Mn²⁺, Zn²⁺, Al²⁺, Cu²⁺, Ni²⁺, Ba²⁺, Sr²⁺, or others, and mixtures thereof. In some embodiments, magnesium, calcium or mixtures thereof, can be present extracellularly at approximately 1.5 mM and 1 mM, respectively. Mixtures of two or more divalent cations can be used in combinations amounting to total concentrations of about 0.9, 2, 4, 5, 6, 7, 8, 9, 10, 12, 15, 20, 45, 65, 90, 130, 170, 220, 280, 320, 350, 500, 750, 1000, 1500 mM, etc., and up to about 2M.

In certain preferred embodiments, the counterion can include PO₄, Cl, OH, CO₂, or mixtures thereof. In other embodiments, the formulations may cause DNA to form particulate or precipitates with size distributions where the mean sizes, or the 80% particles, are in excess of about 0.1, 0.3, 0.5, 1, 3, 5, 8, 15, 20, 35, 50, 70 or 100 microns. Size of such particulates may be evaluated by centrifugation, flow cytometry analysis, propydium iodide or similar dye labeling, or dynamic light scattering.

A pharmaceutical composition comprising BHT-3021 can be incorporated into a variety of formulations for therapeutic administration. More particularly, a combination of the present invention can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of BHT-3021 can be achieved in various ways, including oral, buccal, parenteral, intravenous, intradermal, subcutaneous, intramuscular, transdermal, intrarectal, intravaginal, etc., administration. Moreover, the compound can be administered in a local rather than systemic manner, for example, in a depot or sustained release formulation. In a preferred embodiment, the self-vector is administered intramuscularly.

Formulations suitable for use in the present invention are found in Remington: The Science and Practice of Pharmacy, 21st Ed., University of the Sciences in Philadelphia (USIP), Lippincott Williams & Wilkins (2005), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In some embodiments, the self-vector or DNA plasmid vector can be formulated for intramuscular, subcutaneous, or parenteral administration by injection, e.g., by bolus injection or continuous infusion. For injection, BHT-3021 can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, the self-vector can be formulated in aqueous solutions, for example, in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, BHT-3021 can be readily formulated by combining the inhibitory agent with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

C. Methods of Administration

In some embodiments, the present invention provides a method of treating, reducing, preventing, inhibiting, e.g., the severity and or symptoms of IDDM in a subject comprising administering to the subject a self-vector or DNA plasmid vector of SEQ ID NO:1 (BHT-3021). The self-vector can be administered in a pharmaceutically acceptable carrier. In some embodiments, the self-vector BHT-3021 is administered in a pharmaceutically acceptable carrier or excipient comprising calcium at a concentration about equal to physiological levels (e.g., about 0.9 mM). In some embodiments, the self-vector BHT-3021 is administered in a pharmaceutically acceptable carrier or excipient comprising a divalent cation at a concentration greater than physiological levels. In some embodiments, the divalent cation is calcium. In some embodiments, the calcium is at a concentration greater than about 2 mM; in some embodiments, the calcium is at a concentration of about 5.4 mM. In some embodiments, the self-vector BHT-3021 is endotoxin-free. In some embodiments, the self-vector BHT-3021 is administered intramuscularly.

A wide variety of methods exist to deliver polynucleotide to subjects, as defined herein. For example, the polynucleotide encoding a self-polypeptide can be formulated with cationic polymers including cationic liposomes. Other liposomes also represent effective means to formulate and deliver self-polynucleotide. Alternatively, the DNA can be incorporated into a viral vector, viral particle, or bacterium for pharmacologic delivery. Viral vectors can be infection competent, attenuated (with mutations that reduce capacity to induce disease), or replication-deficient. Methods utilizing DNA to prevent the deposition, accumulation, or activity of pathogenic self proteins may be enhanced by use of viral vectors or other delivery systems that increase humoral responses against the encoded self-protein. In other embodiments, the DNA can be conjugated to solid supports including gold particles, polysaccharide-based supports, or other particles or beads that can be injected, inhaled, or delivered by particle bombardment (ballistic delivery). Methods for delivering nucleic acid preparations are known in the art. See, e.g.; U.S. Pat. Nos. 5,399,346, 5,580,859, and 5,589,466.

A number of viral based systems have been developed for transfer into mammalian cells. For example, retroviral systems have been described (U.S. Pat. No. 5,219,740; Miller et al., Biotechniques 7:980-990, 1989; Miller, Human Gene Therapy 1:5-14, 1990; Scarpa et al., Virology 180:849-852, 1991; Burns et al., Proc. Natl. Acad. Sci. USA 90:8033-8037, 1993; and, Boris-Lawrie and Temin, Cur. Opin. Genet. Develop. 3:102-109, 1993). A number of adenovirus vectors have also been described, see e.g., (Haj-Ahmad et al., J. Virol. 57:267-274, 1986; Bett et al., J. Virol. 67:5911-5921, 1993; Mittereder et al., Human Gene Therapy 5:717-729, 1994; Seth et al., J. Virol. 68:933-940, 1994; Barr et al., Gene Therapy 1:51-58, 1994; Berkner, BioTechniques 6:616-629, 1988; and, Rich et al., Human Gene Therapy 4:461-476, 1993). Adeno-associated virus (AAV) vector systems have also been developed for nucleic acid delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., Molec. Cell. Biol. 8:3988-3996, 1988; Vincent et al., Vaccines 90 (Cold Spring Harbor Laboratory Press) 1990; Carter, Current Opinion in Biotechnology 3:533-539, 1992; Muzyczka, Current Topics in Microbiol. And Immunol. 158:97-129, 1992; Kotin, Human Gene Therapy 5:793-801, 1994; Shelling et al., Gene Therapy 1:165-169, 1994; and, Zhou et al., J. Exp. Med. 179:1867-1875, 1994).

The polynucleotide of this invention can also be delivered without a viral vector. For example, the molecule can be packaged in liposomes prior to delivery to the subject. Lipid encapsulation is generally accomplished using liposomes which are able to stably bind or entrap and retain nucleic acid. For a review of the use of liposomes as carriers for delivery of nucleic acids, see, e.g., Hug et al., Biochim. Biophys. Acta. 1097:1-17, 1991; Straubinger et al., in Methods of Enzymology, Vol. 101, pp. 512-527, 1983.

The parameters of different treatment and maintenance regimens, e.g., defined by a combination of dose amount, dosing frequency and dosing period, can be adjusted based on the ranges of dose, frequency and time period described herein. Therapeutic regimens will generally differ from maintenance regimens in delivering a higher level of the DNA self-vector (e.g., by delivering a higher dose more often or for a longer period) to the patient in order to improve and stabilize disease symptoms. Supplemental or maintenance regimens deliver a lower level of the DNA self-vector to the patient in order to maintain stabilizes symptoms and prevent relapse.

Therapeutically effective amounts of self-vector are in the range of about 0.3 mg to about 6 mg. For example, a therapeutic amount of self-vector is in the range of about 1 mg to 3 mg, for example, in doses of about 1 mg, 1.5 mg, 2 mg, 2.5 mg, 3 mg per administration. The dosing can be adjusted to higher or lower doses, as desired or necessary, over the course of treatment. For example, a therapeutic regimen can start out with a higher dose, e.g., 6 mg/administration or 3 mg/administration, and then change to administration of a lower dose, e.g., 2 mg, 1 mg or 0.3 mg per administration. In some embodiments, the dosing amount is maintained at a constant level throughout the course of treatment.

With respect to frequency of administration or dosing, the self-vector can administered, e.g., weekly, bi-weekly (i.e., every other week or twice monthly) or monthly to achieve a therapeutic effect. In some embodiments, a therapeutic regimen is followed by a maintenance regimen, for example, after a desirable therapeutic end point is achieved. The frequency of administration of the self-vector in a maintenance regimen can be less often than during a therapeutic regimen. For example, the frequency of dosing during a maintenance regimen can be monthly, every other month, semi-annually (i.e., twice a year) or annually as a maintenance dose. Alternative treatment regimens may be developed and may range from daily, to weekly, monthly, to every other month, to yearly, to a one-time administration depending upon the severity of the disease, the age of the patient, the self-polypeptide or polypeptides being administered, and such other factors as would be considered by the ordinary treating physician. The frequency can be adjusted to be more or less frequent, as needed or desired, over the time period of treatment of the patient. For example, initial therapeutic dosing can be more frequent, and the frequency of administration decreased, e.g., when a therapeutic end point is achieved or when transitioning into a maintenance regimen. The frequency of dosing can be increased if the severity of the disease increases and decreased if the severity of disease decreases or if the patient is stabilized.

With respect to the period of dosing or administration of the DNA self-vector, the DNA self-vector can be administered for a period of weeks, months, years, or the life of the patient. In some embodiments, the DNA self-vector is administered over a time period of 4, 5, 6, 7, 8, 9, 10, 11 or 12 weeks. In some embodiments, the DNA self-vector is administered over a time period of 3, 4, 5, 6, 7, 8, 9, 10, 11 or 12 months. In some embodiments, the DNA self-vector is administered over a time period of 1, 2, 3, 4, 5 or more years. In some embodiments, the DNA self-vector is administered until a desired therapeutic end point is reached and maintained, or for the rest of the life of the patient.

In one embodiment, the polynucleotide is delivered by intramuscular (“IM”) injection. For IM administration, the self-vector is formulated in a pharmaceutically acceptable carrier in a concentration sufficient to dissolve the vector. For example, the self-vector can be prepared in a liquid, physiologically acceptable carrier in a concentration of about 1.5 mg/ml to about 3 mg/ml, for example, about 2 mg/ml. The self-vector is injected in a volume sufficient to deliver the vector without undesirable side effects, for example, a volume of about 2 ml or less is injected at a single site, for example, a volume of about 1.5 ml, 1 ml, 0.5 ml or less is injected at a single site. In some embodiments the full dose of the self-vector is delivered at, i.e., divided between, two or more sites.

By way of providing non-limiting examples, following are exemplary regimens for the treatment and maintenance of IDDM in a patient using the self-vector of the invention. In a first example, the self-vector is administered intramuscularly in a dose of, e.g., 0.3 to 6 mg/administration weekly for 12 weeks, then in a dose of, e.g., 0.3 to 6 mg/administration every other week (i.e., twice monthly) for 6-12 weeks, followed by a once yearly maintenance dose of, e.g., 0.3 to 6 mg/administration. In a second example, the self-vector is administered intramuscularly in a dose of, e.g., 0.3 to 6 mg/administration every other week for a period of 6-12 months. In a third example, the self-vector is administered intramuscularly in a dose of, e.g., 0.3 to 6 mg/administration every other week for 6-12 weeks, followed by once monthly maintenance doses of, e.g., 0.3 to 6 mg/administration for a period of 6-12 months. In some embodiments the administered dose is 1 mg, 2 mg, or 3 mg.

A regimen can be repeated, e.g., 2, 3, 4, 5 or more times as necessary. For example, a treatment regimen can be repeated, e.g., sequentially, semi-annually, or annually, as needed, before introducing the patient to a maintenance regimen. In another example, the patient is subjected to a treatment regimen until a desired therapeutic endpoint is reached, and then subject to a maintenance regimen that is repeated, e.g., sequentially, semi-annually, or annually, as needed.

In other variations, the polynucleotide is delivered intranasally, orally, subcutaneously, intradermally, intravenously, mucosally, impressed through the skin, or attached to gold particles delivered to or through the dermis (see, e.g., WO 97/46253). Alternatively, nucleic acid can be delivered into skin cells by topical application with or without liposomes or charged lipids (see e.g. U.S. Pat. No. 6,087,341). Yet another alternative is to deliver the nucleic acid as an inhaled agent. The polynucleotide can be formulated in phosphate buffered saline with physiologic levels of calcium (0.9 mM). Alternatively, the polynucleotide is formulated in solutions containing higher quantities of Ca⁺⁺, e.g., between 1 mM and 2M. The polynucleotide may be formulated with other cations such as zinc, aluminum, and others. Alternatively, or in addition, the polynucleotide may be formulated either with a cationic polymer, cationic liposome-forming compounds, or in non-cationic liposomes. Examples of cationic liposomes for DNA delivery include liposomes generated using 1,2-bis(oleoyloxy)-3-(trimethylammionio) propane (DOTAP) and other such molecules.

Prior to delivery of the polynucleotide, the delivery site can be preconditioned by treatment with bupivicane, cardiotoxin or another agent that may enhance the subsequent delivery of the polynucleotide. Such preconditioning regimens are generally delivered 12 to 96 hours prior to delivery of therapeutic polynucleotide; more frequently 24 to 48 hours prior to delivery of the therapeutic polynucleotide. Alternatively, no preconditioning treatment is given prior to polynucleotide therapy.

The self-vector can be administered in combination with other substances, such as, for example, pharmacological agents, adjuvants, cytokines, or vectors encoding cytokines. Furthermore, to avoid the possibility of eliciting unwanted anti-self cytokine responses when using cytokine codelivery, chemical immunomodulatory agents such as the active form of vitamin D3 can also be used. In this regard, 1,25-dihydroxy vitamin D3 has been shown to exert an adjuvant effect via intramuscular DNA immunization.

C. Co-Administration of Self-Proteins

The present invention also relates to the co-administration of the self-vectors as described above with self-proteins targeted in IDDM, or peptide fragments thereof. The self protein or peptide fragment thereof can be administered with self vector or separately. Thus, any of the treatment and/or maintenance regimens disclosed herein can include co-administration of a self protein.

The self-protein can be any self-protein targeted in IDDM, including, for example, insulin, insulin B chain, proinsulin, and preproinsulin; tyrosine phosphatase IA-2; IA-2β; glutamic acid decarboxylase (GAD) both the 65 kDa and 67 kDa forms; carboxypeptidase H; heat shock proteins (HSP); glima 38; islet cell antigen 69 KDa (ICA69); p52; two ganglioside antigens (GT3 and GM2-1); islet-specific glucose-6-phosphatase-related protein (IGRP); and an islet cell glucose transporter (GLUT 2). The self protein can be administered as a full length protein or as a peptide fragment comprising an autoantigenic epitope. The peptide fragment can be, for example, 5 to 75 amino acids, or 10 to 50 amino acids in length. In many embodiments, the peptide fragment will be from about 10 to about 25 amino acids in length.

If the self protein is insulin, the insulin can be co-administered in the context of insulin replacement therapy according to methods well known to those of skill in the art. The goal of insulin therapy is to mimic normal insulin levels. Thus, the dose and treatment regimen will be tailored for each patient. Such regimens usually include insulin injection or use of an insulin pump, along with attention to dietary management, typically including carbohydrate tracking, and careful monitoring of blood glucose levels.

Alternatively, the self protein or peptide fragment thereof can be administered with the goal of suppressing the immune response against the self protein. In this context, if the self-protein or peptide fragment thereof is administered separately, one of skill will recognize that any of the formulations, modes of administration, or treatment and maintenance regimens described above for the self vector can be used for the self protein, as well. Thus, a pharmaceutical composition comprising the self protein or fragment thereof can be incorporated into a variety of formulations for therapeutic administration. More particularly, the self protein or peptide fragment can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the self protein or peptide fragment can be achieved in various ways, including oral, buccal, parenteral, intravenous, intradermal, subcutaneous, intramuscular, transdermal, intrarectal, intravaginal, etc., administration. Moreover, the self protein or peptide fragment can be administered in a local rather than systemic manner, for example, in a depot or sustained release formulation. In a typical embodiment, the self protein or peptide fragment thereof is administered intramuscularly or intravenously.

In a typical embodiment, the dose, for intravenous co-administration, is from about 0.1 mg per kilogram of body weight to about 10 mg per kilogram of body weight. Typically, the dose will be from about 0.1 mg to about 3 mg per kilogram of body weight, often from about 0.5 mg to about 1 mg per kilogram of body weight.

### EXAMPLES

The following examples are provided to further illustrate the invention but not to limit its scope. Other variants of the invention will be readily apparent to one of ordinary skill in the art and are encompassed by the appended claims.

### Example 1

### Treatment of Established Hyperglycemia in NOD Mice by DNA Vaccination Using BHT-3021 Formulated with Increasing Ca++ Concentrations

This study investigated whether DNA vaccination with BHT-3021 formulated with increasing concentrations of Ca++ decreased the development of diabetes in NOD mice with established hyperglycemia.

Treatment of female NOD mice began after the mice became hyperglycemic with blood glucose levels reaching 190-250 mg/dl (typically at 15-18 weeks of age) as determined by plasma glucose measurements using the One Touch II meter (Johnson & Johnson, Milpitas, Calif.). Mice with overt clinical pre-diabetes were injected in each quadricep with 0.05 ml of 0.25% bupivicaine-HCL (Sigma, St. Louis, Mo.). Two days later the mice (n=5 per group) were administered intramuscularly 0.10 ml of PBS or BHT-3021 at 250 ug/ml in PBS with different final Ca++ concentrations including: 0.9 mM (1×), 2.7 mM (3×) and 5.4 mM (6×) in each quadricep for a total of 50 ug/animal. DNA preparations (0.2 ml) were formulated at 0.25 mg/ml or 1.5 mg/ml with calcium chloride concentrations ranging from 0.9 mM (1×) to 8.1 mM (9×). Samples were placed at −20° C. approximately one hour after formulation and left overnight at −20° C. The samples were thawed at room temperature prior to injection. Separate samples were spun for 5 minutes in an eppendorf microfuge (13,000 rpm). Supernatants were removed and the pellets were resuspended in Tris-EDTA (TE) and OD₂₆₀ readings were taken to determine the amount of DNA in the pellet. DNA injections were continued weekly for a total of 4 weeks. Mice were tested weekly for glucosuria by Chemstrip (Boehringer Mannheim Co., Indianapolis, Ind.) and diabetes was confirmed by plasma glucose measurement using the One Touch II meter (Johnson & Johnson, Milpitas, Calif.). Progression to diabetes was defined as two consecutive blood glucose measurements greater than 250 mg/dl.

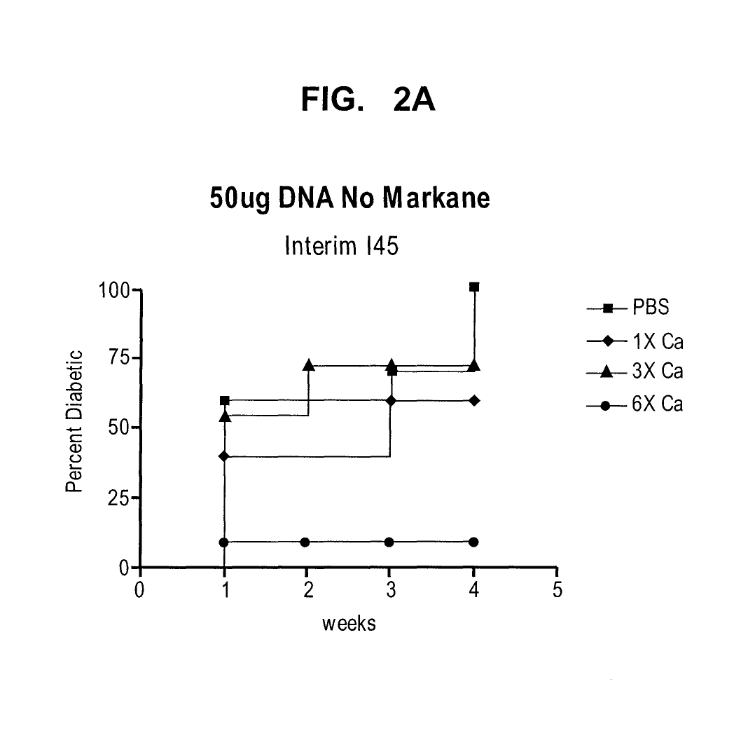

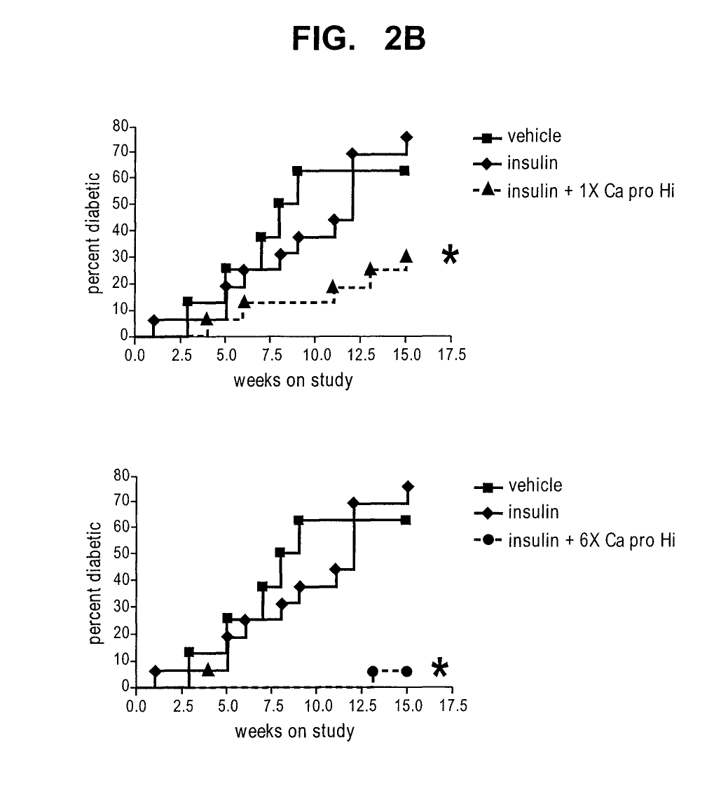

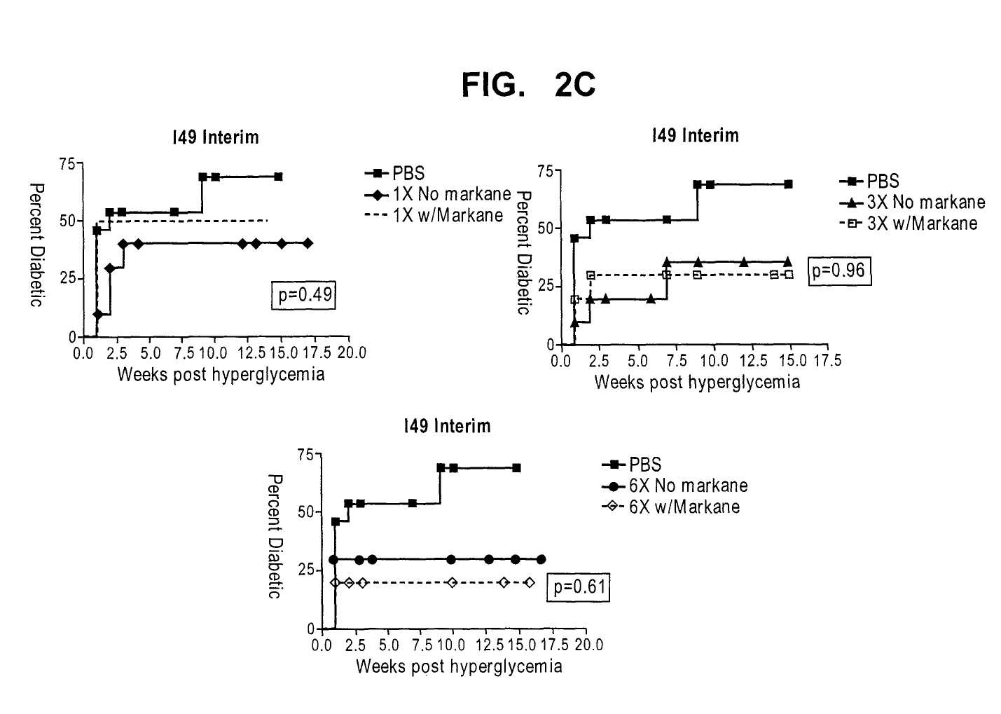

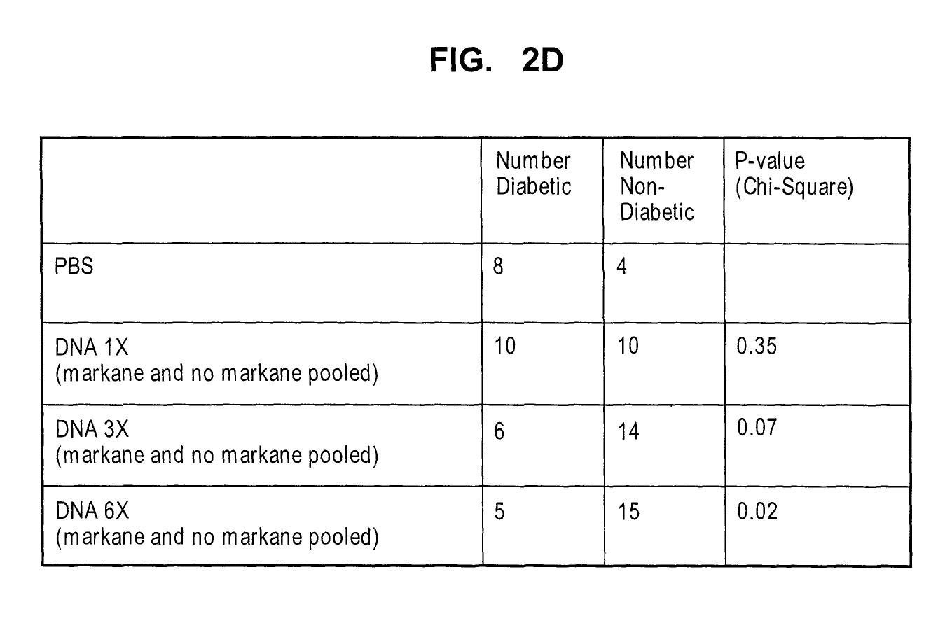

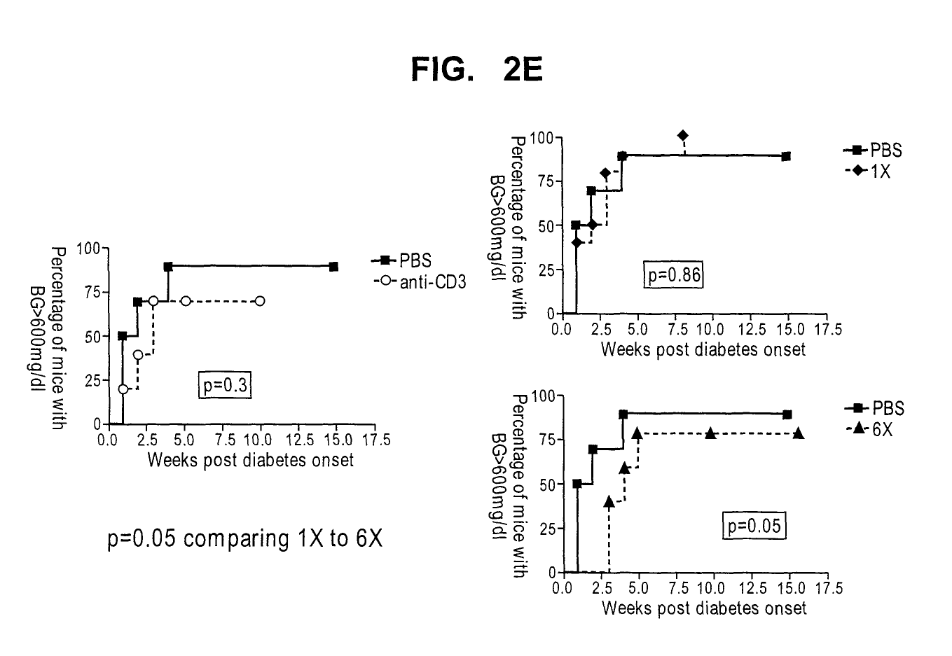

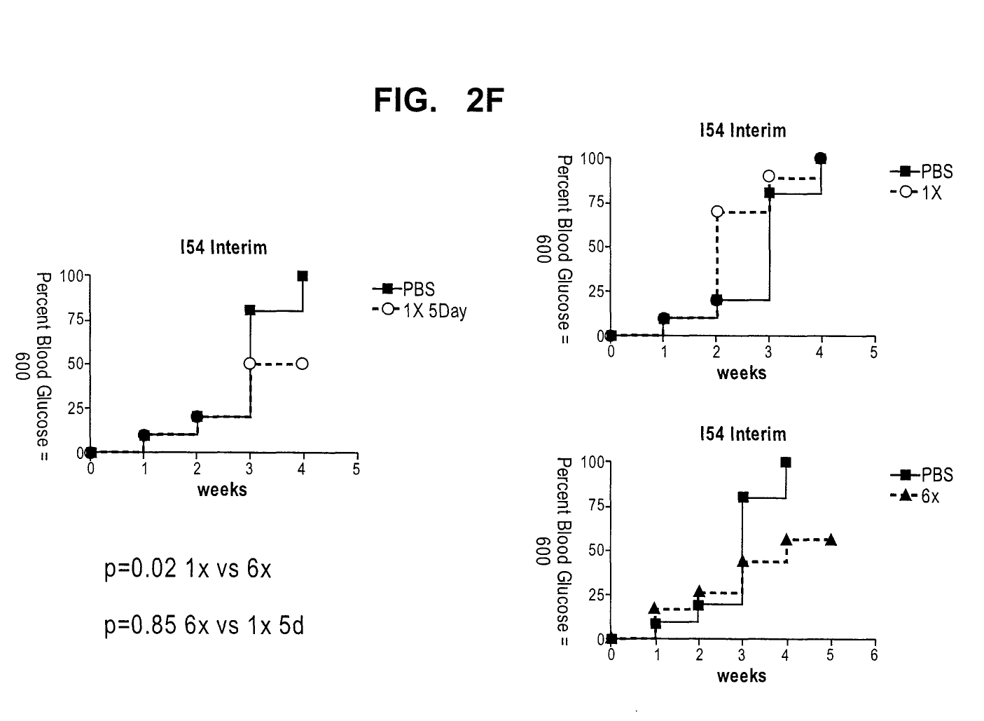

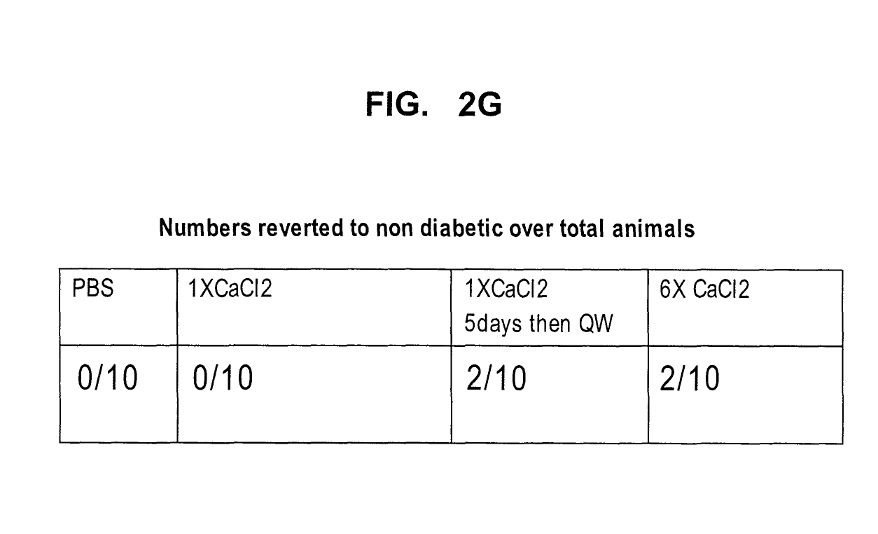

Vaccination with BHT-3021 formulated with 6× Ca++ resulted in a significant reduction in disease progression compared to vaccination with BHT-3021 formulated with 3× or 1× Ca++(FIG. 2A). Similar results were obtained when insulin was co-administered (FIG. 2B). Furthermore, addition of bupivacaine revealed a slight increase in efficacy at 3× and 6× calcium formulations (FIG. 2C). Composite results for diabetic progression with different calcium formulations with or without bupivacaine are summarized in FIG. 2D.

In addition to reducing disease progression, DNA vaccination with a higher calcium formulation also reduced the percentage of mice that obtained blood glucose (BG) levels over 600 mg/dl. Post-diabetes onset, mice were tested for plasma glucose levels using the One Touch II meter (Johnson & Johnson, Milpitas, Calif.). Mice vaccinated with BHT-3021 in 6× calcium showed a significant delay and reduction of high blood glucose levels compared to mice treated with the self-vector in 1× calcium formulations, results that mimicked those obtained with an anti-CD3 positive control (FIG. 2E). A similar reduction in the percentage of mice with high blood glucose levels obtained with 6× calcium formulation was also achieved with a 5 day injection protocol of BHT-3021 with 1× calcium (FIG. 2F). Furthermore, both 6× calcium and 1× calcium injected for 5 days resulted in a reversion of 1/5 of animals with high blood glucose levels to non-diabetic status as compared to no reversion when animals were treated with 1× calcium or PBS control (FIG. 2G). Thus formulation of self-vector plasmids with higher concentrations of calcium significantly increases efficacy of DNA vaccination and can substitute for more frequent dosing regimes.

### Example 2

### Reduction in Antibodies to Insulin in Patients Treated with a DNA Vector Encoding Proinsulin (BHT-3021)

This study investigated whether treatment of patients having IDDM with BHT-3021 reduced the level of anti-insulin antibody titers in the patients.

In a phase 1/2 trial, type 1 diabetic patients who were positive for anti-insulin antibodies at baseline (week 0) were treated with 12 weekly intramuscular 1 mg injections of a proinsulin encoding DNA plasmid vector (BHT-3021) constructed from the pBHT1 plasmid backbone. Each patient was also taking insulin. The plasmid vector was delivered in a pharmaceutically acceptable carrier containing a physiological concentration of calcium (about 0.9 mM). Antibody titers to three pancreatic autoantigens were measured at weeks 0, 2, 4, 6, 8, and 15 where available. The three antibodies, measured by radioimmunoassay and expressed as radioactivity index units, are antibodies to GAD, ICA512, and insulin (mIAA).

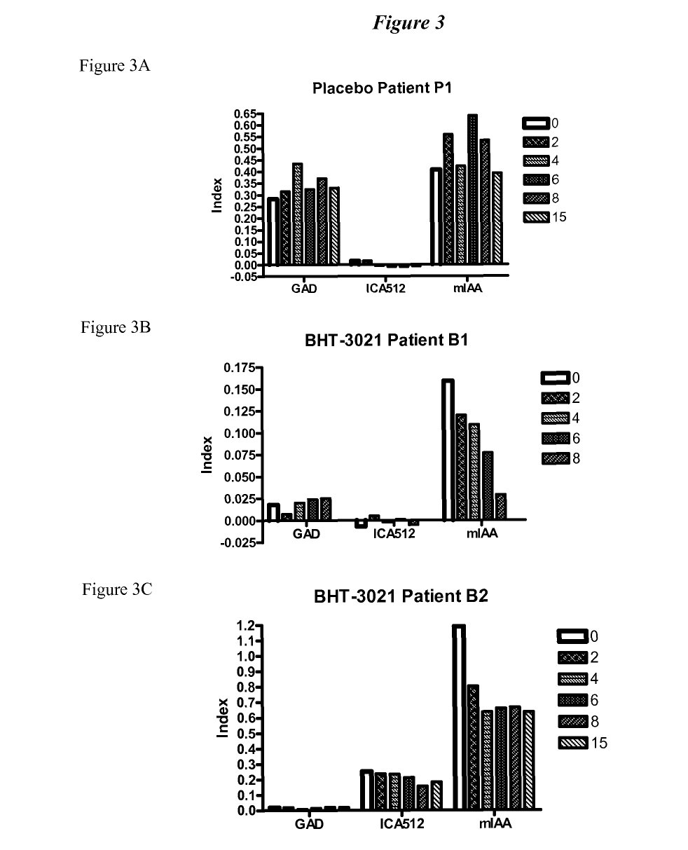

For a patient treated with placebo (saline) injections, positive antibody titers to GAD and insulin were detected at baseline, but those antibody titers did not change with treatment (FIG. 3A). A patient treated with BHT-3021 also had positive antibody titers to GAD and insulin at baseline (FIG. 3B); with treatment, the patient's antibody titers to insulin decreased. Another patient treated with BHT-3021 had positive antibody titers to ICA512 and insulin at baseline (FIG. 3C); with treatment, that patient's antibody titers to insulin decreased. These data demonstrate that BHT-3021 causes antigen-specific immune tolerance as demonstrated by rapid and sustained reductions in anti-insulin titers.

### Example 3

### Preservation of C-Peptide as an Indicator of Beta Cell Function in Patients Treated with a DNA Plasmid Vector Encoding Proinsulin (BHT-3021)

This study investigated whether treatment of patients having IDDM with BHT-3021 preserved the function of β cells.

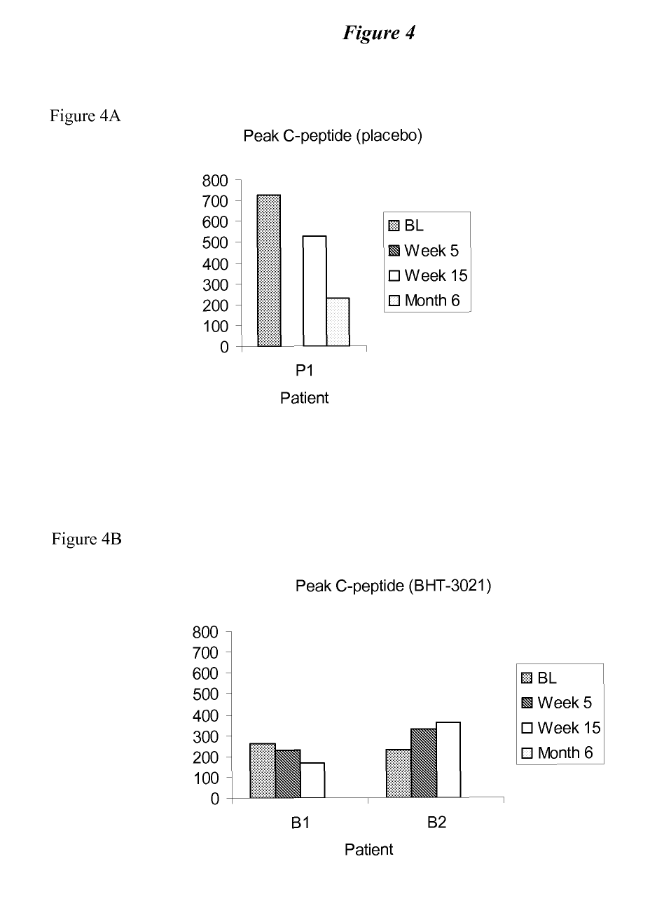

In the same type I diabetic patients represented in Example 2, blood C-peptide levels were determined as measure of residual pancreatic β cell function. Blood C-peptide levels were measured at baseline (BL), week 5, week 15, and month 6, where available. The patient who received placebo (saline) injections exhibited a blood C-peptide level that steadily declined with no treatment (FIG. 4A). The two patients who were treated with BHT-3021, however, exhibited either blood C-peptide levels that declined less rapidly or that increased in value slightly (FIG. 4B), indicating preservation of β cell function.

### Example 4

### Treatment Regimen 1

### Dose (1 mg), Dose Frequency (Weekly) and Dose Period (Twelve Weeks)

BHT-3021 or BHT-placebo was co-administered intramuscularly to human subjects weekly for 12 weeks (Weeks 0 to 11), along with insulin. Approximately 72 subjects were enrolled overall. Evaluation of four dose levels of BHT-3021 was carried out: 0.3 mg, 1 mg, 3 mg, and 6 mg.

BHT-3021 and BHT-placebo were given as intramuscular (IM) injections into the deltoid muscles administered once weekly for 12 weeks. If the subject cannot tolerate an IM injection in the deltoid muscle, then IM injection in the quadriceps muscle was performed. The volumes injected were adjusted based upon the dose level: 0.15 mL for the 0.3 mg dose (i.e., 2 mg/ml), 0.5 mL for the 1 mg dose, 1.5 mL for the 3 mg dose, and 3 mL (two injections) for the 6 mg dose. The 0.15 mL, 0.5 mL, and 1.5 mL volume injections were given into a single muscle site. Injection sites were rotated as necessary. For example, if the drug was injected in the right deltoid in Week 0, the drug was injected in the left deltoid the following week. The 3 mL volume injections for delivering 6 mg of the drug were divided into two 1.5 mL volume injections and were given into two separate muscle sites.

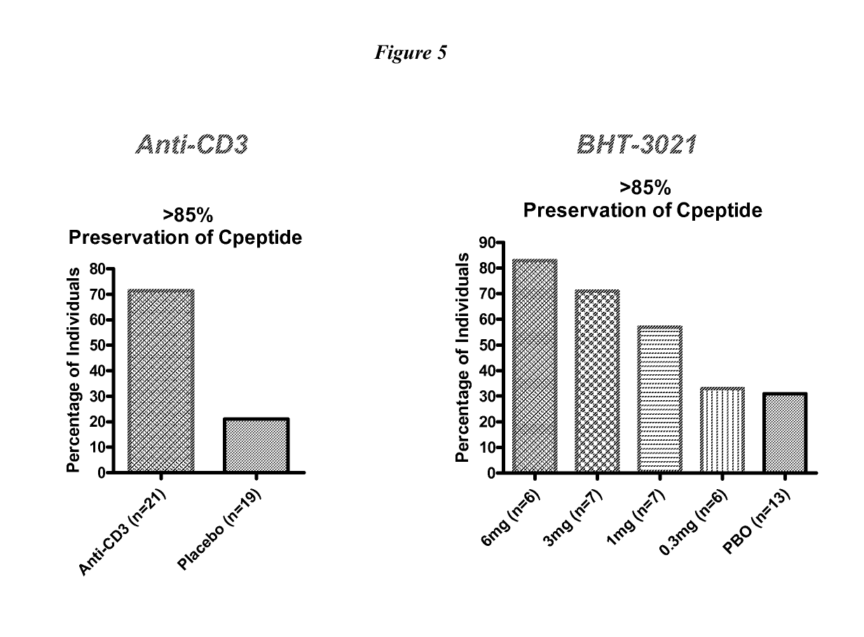

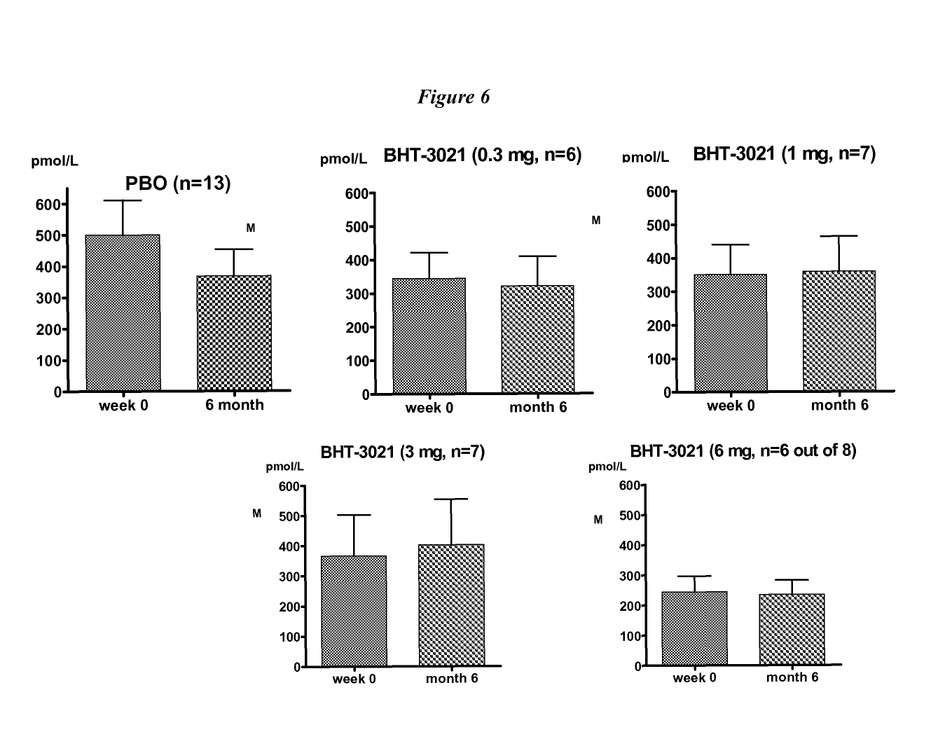

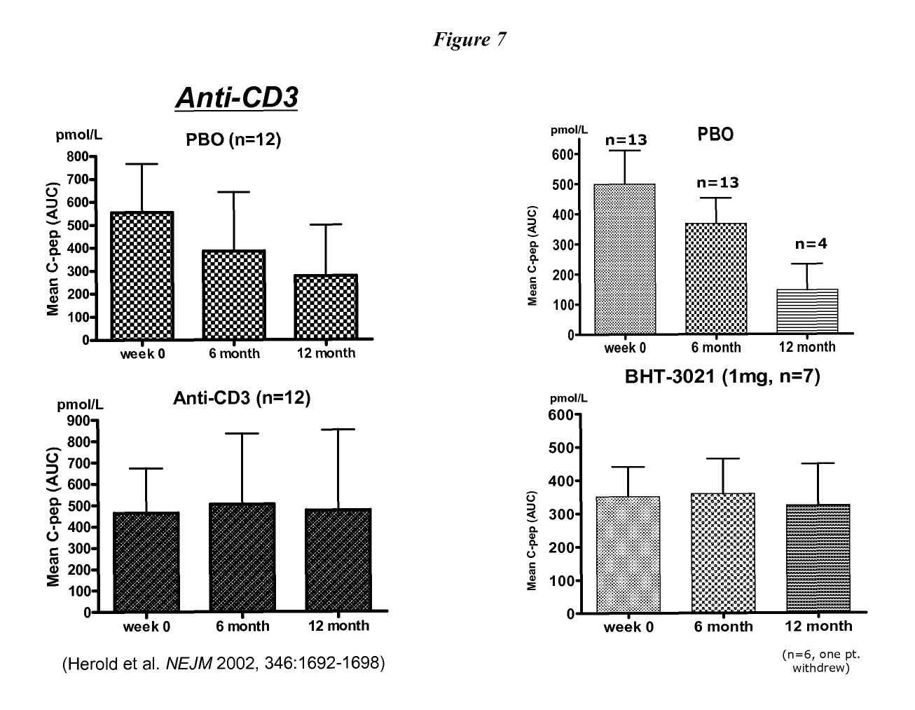

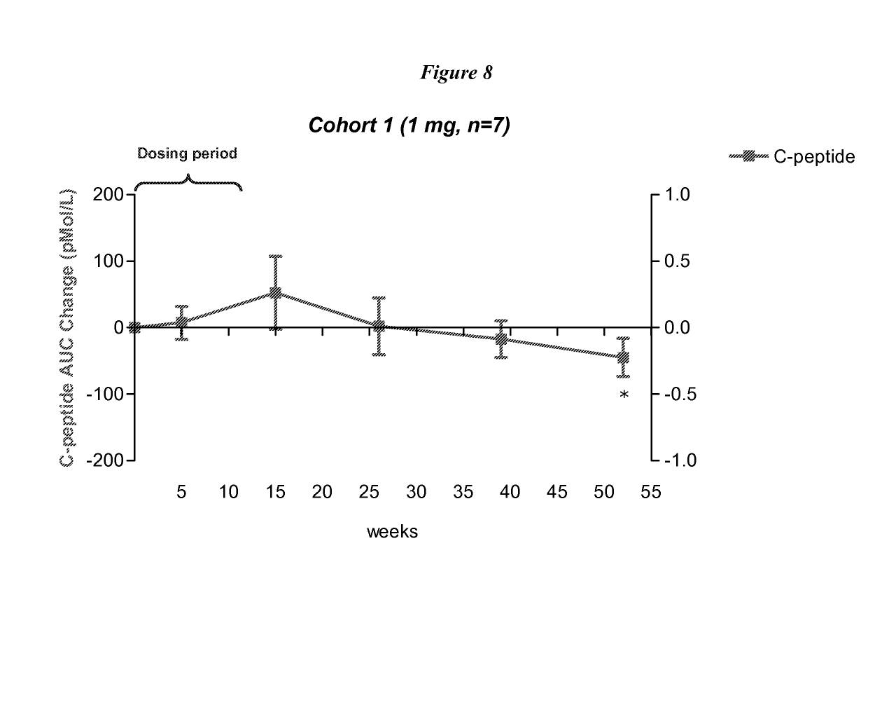

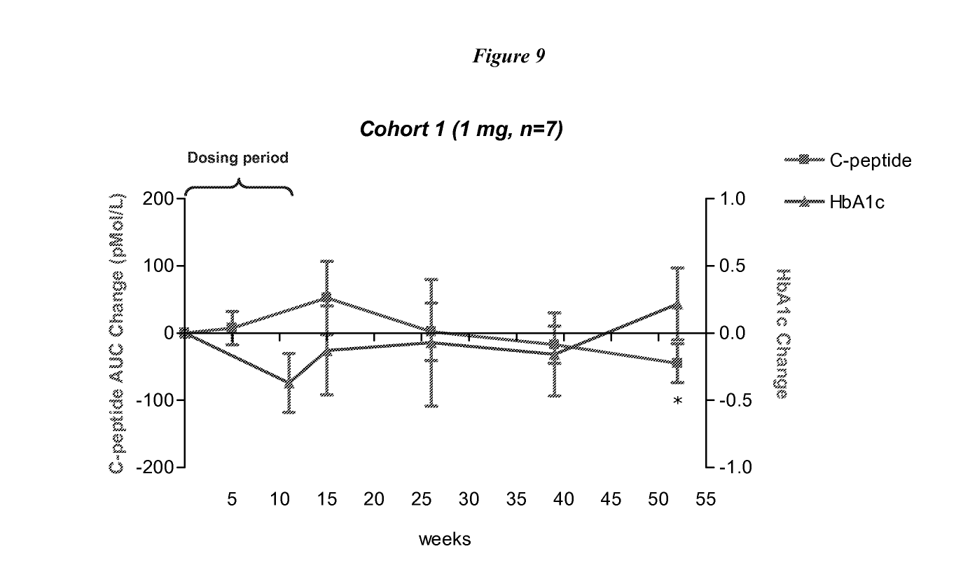

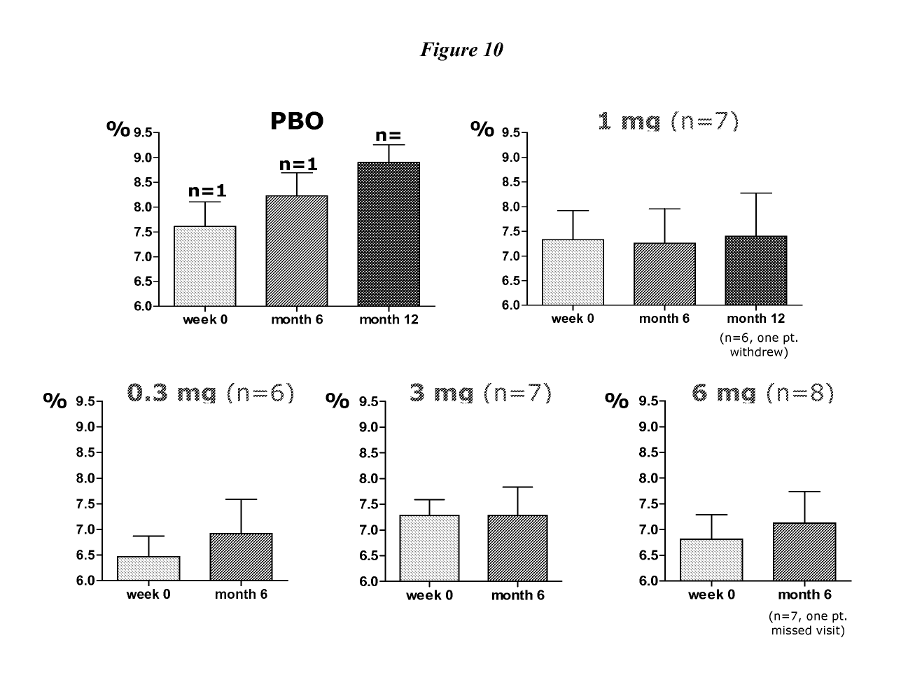

The results of patient evaluations at the 6-month and 12-month time points, as indicated by preservation of C-peptide levels and glycosylated hemoglobin HbA1c levels, are shown in FIGS. 5-9.

### Example 5

### Treatment Regimen 2

### Dose (1 mg), Dose Frequency (Bi-Weekly) and Dose Period (Continuous)

BHT-3021 is co-administered intramuscularly along with insulin to human subjects having IDDM and/or related diseases bi-weekly (i.e, every other week or once every two weeks) for the full period of treatment, e.g., until a desired therapeutic endpoint is achieved or for the life of the patient.

A dose of 1 mg of BHT-3021 in 0.5 mL is given in intramuscular (IM) injections into the deltoid or quadriceps muscles administered bi-weekly for the full period of treatment.

The subject is evaluated over the course of treatment for one or more indicators of severity of the disease. The patient is evaluated for the one or more indicators prior to every administration of the self vector. For example, one or more measures including but not limited to increased or stabilized levels of C-peptide, increased or stabilized levels of glycosylated hemoglobin, decreased hyperglycemia, increased plasma insulin, decreased glucosuria, decreased insulitis, decreased destruction of beta-cells, and decreased presence of autoantibodies are monitored before every administration of the self-vector to determine the efficacious effect for reducing severity of disease. Additional indicators of disease severity for IDDM are known in the art and described herein. A pre-determined therapeutic end point or threshold level of one or more measures is set at the beginning of or during the course of treatment. The threshold levels of the different indices evidencing efficacy are established in the art, e.g., normal or desired levels in the blood, serum or plasma of C-peptide >0.20 pmol/L, glycosylated hemoglobin <=7.0, insulin, sugar between 70 and 180 mg/dL, blood sugar <250 mg/dL, decreased incidence/time of blood sugar <70 mg/dL, etc. Alternatively, the measures are determined in the subject before treatment has commenced, or at a time point during the course of treatment, and compared with measures at a later time point during treatment. When one or more pre-determined therapeutic end points or threshold levels are reached, and maintained for several weeks or months, the physician can decide to either continue or end the dosing period. The dosing administrations can continue as long as needed to achieve the desired therapeutic endpoint. Depending on the patient, the dosing period can be 6 months, 1 year, 1.5 years, 2.0 years, for the life of the patient, or longer or shorter, as judged by a physician.

Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims that follow. All publications or patent documents cited in this specification are incorporated herein by reference as if each such publication or document was specifically and individually indicated to be incorporated herein by reference. Citation of the above publications or documents is not intended as an admission that any of the foregoing is pertinent prior art, nor does it constitute any admission as to the contents or date of these publications or documents.

INFORMAL SEQUENCE LISTING

SEQ ID NO: 1 (BHT-3021)
GCTGCTTCGCGATGTACGGGCCAGATATACGCGTTGACATTGATTATTGA
CTAGTTATTAATAGTAATCAATTACGGGGTCATTAGTTCATAGCCCATAT
ATGGAGTTCCGCGTTACATAACTTACGGTAAATGGCCCGCCTGGCTGACC
GCCCAACGACCCCCGCCCATTGACGTCAATAATGACGTATGTTCCCATAG
TAACGCCAATAGGGACTTTCCATTGACGTCAATGGGTGGAGTATTTACGG
TAAACTGCCCACTTGGCAGTACATCAAGTGTATCATATGCCAAGTACGCC
CCCTATTGACGTCAATGACGGTAAATGGCCCGCCTGGCATTATGCCCAGT
ACATGACCTTATGGGACTTTCCTACTTGGCAGTACATCTACGTATTAGTC
ATCGCTATTACCATGGTGATGCGGTTTTGGCAGTACATCAATGGGCGTGG
ATAGCGGTTTGACTCACGGGGATTTCCAAGTCTCCACCCCATTGACGTCA
ATGGGAGTTTGTTTTGGCACCAAAATCAACGGGACTTTCCAAAATGTCGT
AACAACTCCGCCCCATTGACGCAAATGGGCGGTAGGCGTGTACGGTGGGA
GGTCTATATAAGCAGAGCTCTCTGGCTAACTAGAGAACCCACTGCTTACT
GGCTTATCGAAATTAATACGACTCACTATAGGGAGACCCAAGCTGGCTAG
CGTAAGTATCAAGGTTACAAGACAGGTTTAAGGAGACCAATAGAAACTGG
GCTTGTCGAGACAGAGAAGACTCTTGCGTTTCTGATAGGCACCTATTGGT
CTTACTGACATCCACTTTGCCTTTCTCTCCACAGGCTTAAGCTTATGGCC
TTTGTGAACCAACACCTGTGCGGCTCACACCTGGTGGAAGCTCTCTACCT
AGTGTGCGGGGAACGAGGCTTCTTCTACACACCCAAGACCCGCCGGGAGG
CAGAGGACCTGCAGGTGGGGCAGGTGGAGCTGGGCGGGGGCCCTGGTGCA
GGCAGCCTGCAGCCCTTGGCCCTGGAGGGGTCCCTGCAGAAGCGTGGCAT
TGTGGAACAATGCTGTACCAGCATCTGCTCCCTCTACCAGCTGGAGAACT
ACTGCAACTAGCTCGAGTCTAGAGGGCCCGTTTAAACCCGCTGATCAGCC
TCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAG
GCATGCTGGGGATGCGGTGGGCTCTATGGCTTCTACTGGGCGGTTTTATG
GACAGCAAGCGAACCGGAATTGCCAGCTGGGGCGCCCTCTGGTAAGGTTG
GGAAGCCCTGCAAAGTAAACTGGATGGCTTTCTTGCGGCCAAGGATCTGA
TGGCGCAGGGGATCAAGCTCTGATCAAGAGACAGGATGAGGATGGTTTCG
CATGATTGAACAAGATGGATTGCACGCAGGTTCTCCGGCAGCTTGGGTGG
AGAGGCTATTCGGCTATGACTGGGCACAACAGACAATCGGCTGCTCTGAT
GCCGCCGTGTTCAGGCTGTCAGCGCAGGGGCGCCCGGTTCTTTTTGTCAA
GACCGACCTGTCCGGTGCCCTGAATGAACTGCAAGACGAGGCAGCGCGGC
TATCGTGGCTGGCCACGACGGGCGTTCCTTGCGCAGCTGTGCTCGACGTT
GTCACTGAAGCGGGAAGGGACTGGCTGCTATTGGGCGAAGTGCCGGGGCA
GGATCTCCTGTCATCTCACCTTGCTCCTGCCGAGAAAGTATCCATCATGG
CTGATGCAATGCGGCGGCTGCATACGCTTGATCCGGCTACCTGCCCATTC
GACCACCAAGCGAAACATCGCATCGAGCGAGCACGTACTCGGATGGAAGC
CGGTCTTGTCGATCAGGATGATCTGGACGAAGAGCATCAGGGGCTCGCGC
CAGCCGAACTGTTCGCCAGGCTCAAGGCGAGCATGCCCGACGGCGAGGAT
CTCGTCGTGACCCATGGCGATGCCTGCTTGCCGAATATCATGGTGGAAAA
TGGCAGGTTTTCTGGATTCATCGACTGTGGCCGGCTGGGTGTGGCGGACA
GGTATCAGGACATAGCGTTGGCTACCCGTGATATTGCTGAAGAGCTTGGC
GGCGAATGGGCTGACAGGTTCCTCGTGCTTTACGGTATTGCGGCTCCCGA
TTCGCAGCGCATTGCCTTCTATAGGCTTCTTGACGAGTTCTTCTGAATTA
TTAACGCTTACAATTTCCTGATGCGGTATTTTCTCCTTACGCATCTGTGC
GGTATTTCACACCGCATCAGGTGGCACTTTTCGGGGAAATGTGCGCGGAA
CCCCTATTTGTTTATTTTTCTAAATACATTCAAATATGTATCCGCTCATG
AGACAATAACCCTGATAAATGCTTCAATAATAGCACGTGCTAAAACTTCA
TTTTTAATTTAAAAGGATCTAGGTGAAGATCCTTTTTGATAATCTCATGA
CCAAAATCCCTTAACGTGAGTTTTCGTTCCACTGAGCGTCAGACCCCGTA
GAAAAGATCAAAGGATCTTCTTGAGATCCTTTTTTTCTGCGCGTAATCTG
CTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCG
CAGATACCAAATACTGTTCTTCTAGTGTAGCCGTAGTTAGGCCACCACTT
CAAGAACTCTGTAGCACCGCCTACATACCTCGCTCTGCTAATCCTGTTAC
CAGTGGCTGCTGCCAGTGGCGATAAGTCGTGTCTTACCGGGTTGGACTCA
AGACGATAGTTACCGGATAAGGCGCAGCGGTCGGGCTGAACGGGGGGTTC
GTGCACACAGCCCAGCTTGGAGCGAACGACCTACACCGAACTGAGATACC
TACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGC
CTATGGAAAAACGCCAGCAACGCGGCCTTTTTACGGTTCCTGGCCTTTTG
CTGGCCTTTTGCTCACATGTTCTT

What is claimed is:

1. A method of reducing disease severity in a subject afflicted with insulin dependent diabetes mellitus (IDDM), the method comprising administering intramuscularly to the subject a DNA plasmid vector encoding an self-protein comprising an epitope associated with IDDM, wherein the administration of the DNA plasmid vector is according to a regimen comprising a combination of:
(a) a therapeutically effective amount of the DNA plasmid vector of from 0.3 to 6 mg;
(b) a dose frequency of weekly or bi-weekly dosing; and,
(c) a period of dosing selected from the group consisting of continuous dosing, four (4) weeks, six (6) weeks, twelve (12) weeks, twenty-four (24) weeks, one (1) year, eighteen (18) months, or two (2) years.

2. The method of claim 1, wherein the self-protein is proinsulin.

3. The method of claim 1, wherein the DNA plasmid is BHT-3021.

4. The method of claim 1 wherein the dose of DNA plasmid is from 1 to 3 mg.

5. The method of claim 1 wherein the dose of DNA plasmid is 0.3 mg.

6. The method of claim 1 wherein the dose of DNA plasmid is 1 mg.

7. The method of claim 1 wherein the dose of DNA plasmid is 2 mg.

8. The method of claim 1 wherein the dose of DNA plasmid is 3 mg.

9. The method of claim 1 wherein the dose of DNA plasmid is 6 mg.

10. The method of claim 1 wherein the dose frequency is weekly.

11. The method of claim 1 wherein the dose frequency is bi-weekly.

12. The method of claim 1 wherein the dosing period is continuous.

13. The method of claim 1 wherein the dosing period is four (4) weeks.

14. The method of claim 1 wherein the dosing period is six (6) weeks.

15. The method of claim 1 wherein the dosing period is twelve (12) weeks.

16. The method of claim 1 wherein the dosing period is one year.

17. The method of claim 13, 14, or 15 wherein the regimen further comprises a supplemental regimen comprising a therapeutically effective amount of the DNA plasmid at a subsequent dose frequency of every other week dosing for a dosing period of six (6) weeks.

18. The method of claim 17 wherein the regimen and supplemental regimen are repeated annually.

19. The method of claim 1, wherein a reduction in the severity of IDDM in the subject is indicated by one or more measures selected from the group consisting of increased or stabilized levels of C-peptide, increased or stabilized levels of glycosylated hemoglobin, decreased hyperglycemia, increased plasma insulin, decreased glucosuria, decreased insulitis, decreased destruction of beta-cells, and decreased presence of autoantibodies.

20. The method of claim 1, wherein the subject is a human.

21. A method of reducing disease severity in a subject afflicted with insulin dependent diabetes mellitus (IDDM), the method comprising administering intramuscularly to the subject a DNA plasmid vector of SEQ ID NO:1 (BHT-3021), wherein the administration of the DNA plasmid vector is according to a regimen comprising administering a dose of 0.3 to 6 mg of the DNA plasmid vector weekly for 12 weeks followed by administering a dose of 0.3 to 6 mg of the DNA plasmid vector bi-weekly for 6 weeks; wherein the regimen is repeated once per year.

22. The method of claim 21 wherein the dose of DNA plasmid is 1 mg.

23. The method of claim 21 wherein the dose of DNA plasmid is 2 mg.

24. The method of claim 21 wherein the dose of DNA plasmid is 3 mg.

25. A method of reducing disease severity in a subject afflicted with insulin dependent diabetes mellitus (IDDM), the method comprising administering intramuscularly to the subject a DNA plasmid vector of SEQ ID NO:1 (BHT-3021), wherein the administration of the DNA plasmid vector is according to a regimen comprising administering a dose of 0.3 to 6 mg of the DNA plasmid vector bi-weekly for the life of the patient.

26. The method of claim 25 wherein the dose of DNA plasmid is 1 mg.

27. The method of claim 25 wherein the dose of DNA plasmid is 2 mg.

28. The method of claim 25 wherein the dose of DNA plasmid is 3 mg.

29. A method of reducing disease severity in a subject afflicted with insulin dependent diabetes mellitus (IDDM), the method comprising administering intramuscularly to the subject a DNA plasmid vector of SEQ ID NO:1 (BHT-3021), wherein the administration of the DNA plasmid vector is according to a regimen comprising administering a dose of 0.3 to 6 mg of the DNA plasmid vector bi-weekly for 6 weeks followed by administering a dose of 0.3 to 6 mg of the DNA plasmid vector monthly for the life of the patient.

30. The method of claim 29 wherein the dose of DNA plasmid is 1 mg.

31. The method of claim 29 wherein the dose of DNA plasmid is 2 mg.

32. The method of claim 29 wherein the dose of DNA plasmid is 3 mg.
